(19) United States
(12) Patent Application Publication
(10) Pub. No.: US 2008/0221100 A1
(43) Pub. Date: Sep. 11, 2008

(54) SOLUBLE EPOXIDE HYDROLASE INHIBITORS

(75) Inventors: Richard D. Gless, Oakland, CA (US); Sampath Kumar Anandan, Fremont, CA (US); Bhaskar R. Aavula, Pleasanton, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 11/875,673

(22) Filed: Oct. 19, 2007

Related U.S. Application Data
(60) Provisional application No. 60/853,226, filed on Oct. 20, 2006.
(60) Provisional application No. 60/894,639, filed on Mar. 13, 2007.

(51) Int. Cl. A61K 31/4468; C07D 413/06; C07D 211/58; A61K 31/4545; A61P 9/00; A61P 11/00; A61P 3/10; A61P 29/00; A61K 31/454; C07D 401/06; A61K 31/5377

(52) U.S. Cl. 514/235.5; 544/130; 546/224; 546/194; 546/210; 514/329; 514/318; 514/326

(57) ABSTRACT

Disclosed are urea compounds, stereoisomer, or pharmaceutical acceptable salt thereof, and compositions that inhibit soluble epoxide hydrolase (sEH), methods for preparing the compounds and compositions, and methods for treating patients with such compounds and compositions. The compounds, compositions, and methods are useful for treating a variety of sEH mediated diseases, including hypertensive, cardiovascular, inflammatory, pulmonary, and diabetic-related diseases.

### CROSS-REFERENCE TO RELATED APPLICATIONS

This application claims the benefit under 35 U.S.C. §119(e) of provisional Patent Application Ser. Nos. 60/853,226, filed on Oct. 20, 2006, and 60/894,639, filed on Mar. 13, 2007, both of which are incorporated herein by reference in their entirety.

### BACKGROUND

1. Field of the Invention

This invention relates to the field of pharmaceutical chemistry. Provided herein are urea compounds that inhibit soluble epoxide hydrolase (sEH), pharmaceutical compositions containing such compounds, methods for preparing the compounds and formulations, and methods for treating patients with such compounds and compositions. The compounds, compositions, and methods are useful for treating a variety of sEH mediated diseases, including hypertensive, cardiovascular, inflammatory, pulmonary, and diabetic-related diseases.

2. State of the Art

The arachidonate cascade is a ubiquitous lipid signaling cascade in which arachidonic acid is liberated from the plasma membrane lipid reserves in response to a variety of extra-cellular and/or intra-cellular signals. The released arachidonic acid is then available to act as a substrate for a variety of oxidative enzymes that convert arachidonic acid to signaling lipids that play critical roles, for example, in inflammation. Disruption of the pathways leading to the lipids remains an important strategy for many commercial drugs used to treat a multitude of inflammatory disorders. For example, non-steroidal anti-inflammatory drugs (NSAIDs) disrupt the conversion of arachidonic acid to prostaglandins by inhibiting cyclooxygenases (COX1 and COX2). New asthma drugs, such as SINGULAIR™ disrupt the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in endothelium (cells that make up arteries and vascular beds), kidney, and lung. In contrast to many of the end products of the prostaglandin and leukotriene pathways, the EETs have a variety of anti-inflammatory and anti-hypertensive properties and are known to be potent vasodilators and mediators of vascular permeability.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been found to significantly reduce blood pressure in hypertensive animals (see, e.g., Yu et al., Circ. Res. 87:992-8 (2000) and Sinal et al., J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see. Schmelzer et al., Proc. Nat'l Acad. Sci. USA 102(28):9772-7 (2005)).

Various small molecule compounds have been found to inhibit sEH and elevate EET levels (Morisseau et al., Annu. Rev. Pharmacol. Toxicol. 45:311-33 (2005)). Heretofore, such small molecules typically included an adamantyl urea moiety or a phenyl or substituted phenyl moiety. While possessing good inhibitory activity, the availability of more potent compounds capable of inhibiting sEH and its inactivation of EETs would be highly desirable for treating a wide range of disorders that arise from inflammation and hypertension or that are otherwise mediated by sEH.

### SUMMARY OF THE INVENTION

This invention is directed to the unexpected discovery that halo-substitution on the phenyl group of the phenylurea moiety provides significant enhancement of the inhibitory activity of these compounds regardless of whether the halo group is attached directly to the phenyl moiety or to an alkyl group bound to the phenyl moiety. Specifically, this invention is directed to such compounds and their pharmaceutical compositions, to their preparation, and to their uses for treating diseases mediated by soluble epoxide hydrolase (sEH). In accordance with one aspect of the invention, provided are compounds having formula I or a stereoisomer, tautomer, or pharmaceutically acceptable salt thereof:

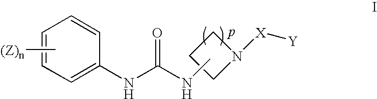

wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3;
provided that when X is C═O and (Z)ₙ is 4-fluoro, Y is not methyl or ethoxy,
provided that when X is SO₂ and (Z)ₙ is 3-fluoro, Y is not 4-tert-butylphenyl, 4-acetylphenyl, 3-methylesterphenyl, or 4-acetylaminophenyl, and
provided that

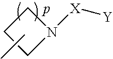

is not

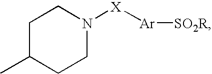

wherein X is as defined herein, Ar is arylene, substituted arylene, heteroarylene or substituted heteroarylene, and R is amino or substituted amino.

In some embodiments, provided are compounds having formula II or a stereoisomer, tautomer, or pharmaceutically acceptable salt thereof:

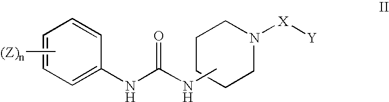

wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3;
provided that when X is C═O and (Z)ₙ is 4-fluoro, Y is not methyl or ethoxy,
provided that when X is SO₂ and (Z)ₙ is 3-fluoro, Y is not 4-tert-butylphenyl, 4-acetylphenyl, 3-methylesterphenyl, or 4-acetylaminophenyl, and
provided that

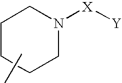

is not

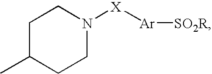

wherein X is as defined herein, Ar is arylene, substituted arylene, heteroarylene or substituted heteroarylene, and R is amino or substituted amino.

In some embodiments, provided are compounds having formula VII or a stereoisomer, tautomer, or pharmaceutically acceptable salt thereof:

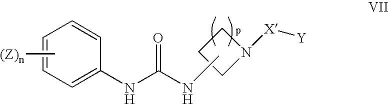

wherein:
X′ is S or SO;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3.

In some embodiments, provided are compounds having formula VIII or a stereoisomer, tautomer, or pharmaceutically acceptable salt thereof:

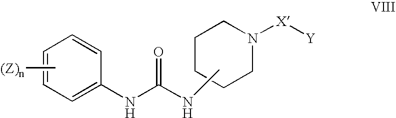

wherein:
X′ is S or SO;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3.

These and other embodiments of the present invention are further described in the text that follows.

### DETAILED DESCRIPTION OF VARIOUS EMBODIMENTS

### Definitions

As used herein, the following definitions shall apply unless otherwise indicated.

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“Chronic Obstructive Pulmonary Disease” or “COPD” is also sometimes known as “chronic obstructive airway disease,” “chronic obstructive lung disease,” and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forcibly expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds), 14th Ed., 1998, McGraw-Hill, New York, pp. 1451-1460 (hereafter, “Harrison's Principles of Internal Medicine”). As the names imply, “obstructive pulmonary disease” and “obstructive lung disease” refer to obstructive diseases, as opposed to restrictive diseases. These diseases particularly include COPD, bronchial asthma, and small airway disease.

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months, a year, for two years, etc.

“Small airway disease” refers to diseases where airflow obstruction is due, solely or predominantly to involvement of the small airways. These are defined as airways less than 2 mm in diameter and correspond to small cartilaginous bronchi, terminal bronchioles, and respiratory bronchioles. Small airway disease (SAD) represents luminal obstruction by inflammatory and fibrotic changes that increase airway resistance. The obstruction may be transient or permanent.

“Interstitial lung diseases (ILDs)” are restrictive lung diseases involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with such restrictive lung disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

“Diabetic neuropathy” refers to acute and chronic peripheral nerve dysfunction resulting from diabetes.

“Diabetic nephropathy” refers to renal diseases resulting from diabetes.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NRC(O)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NRC(S)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR—SO₂NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR¹²)NR¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, —NR—C(O)O-substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spiro groups such as spiro[4.5]dec-8-yl:

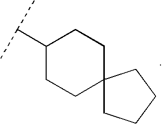

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group, provided that at least one R¹³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein. “Fluoroalkyl” refers to haloalkyl groups wherein the halo group is fluoro and includes, for example, fluoromethyl, trifluoromethyl, 2,2,2-trifluoroethyl and the like.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocyclyl.

“Substituted heterocyclyloxy refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cycloalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Therapeutically effective amount” refers to that amount of an active compound as disclosed in embodiments of the present invention that is effective for treating or preventing the disease.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, the present invention provides a compound of formula I or a stereoisomer, tautomer, or pharmaceutically acceptable salt thereof:

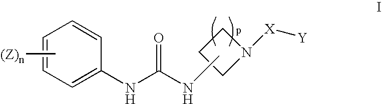

wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3;
provided that when X is C═O and (Z)ₙ is 4-fluoro, Y is not methyl or ethoxy, and
provided that when X is SO₂ and (Z)ₙ is 3-fluoro, Y is not 4-tert-butylphenyl, 4-acetylphenyl, 3-methylesterphenyl, or 4-acetylaminophenyl, and
provided that

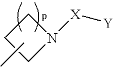

is not

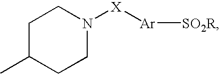

wherein X is as defined herein, Ar is arylene, substituted arylene, heteroarylene or substituted heteroarylene, and R is amino or substituted amino.

In some embodiments, the present invention provides a compound of formula II or a stereoisomer, tautomer, or pharmaceutically acceptable salt thereof:

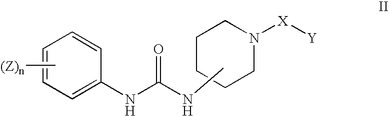

wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl; and
n is an integer equal to 1, 2, or 3;
provided that when X is C═O and (Z)ₙ is 4-fluoro, Y is not methyl or ethoxy, and
provided that when X is SO₂ and (Z)ₙ is 3-fluoro, Y is not 4-tert-butylphenyl, 4-acetylphenyl, 3-methylesterphenyl, or 4-acetylaminophenyl, and
provided that

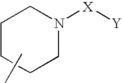

is not

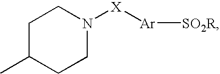

wherein X is as defined herein, Ar is arylene, substituted arylene, heteroarylene or substituted heteroarylene, and R is amino or substituted amino.

In some embodiments, provided is a compound of formula II wherein Z is independently selected from the group consisting of halogen and haloalkyl, and n is an integer equal to 1 to 2.

In some embodiments, the present invention provides a compound of formula III, a stereoisomer, tautomer, or pharmaceutically acceptable salt thereof:

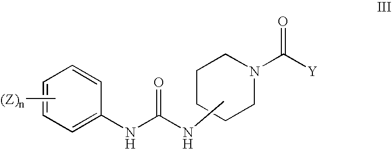

wherein:
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of fluoro and fluoroalkyl; and
n is an integer equal to 1, 2, or 3;
provided that

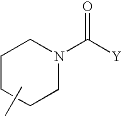

is not

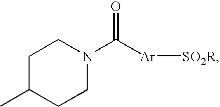

wherein Ar is arylene, substituted arylene, heteroarylene or substituted heteroarylene, and R is amino or substituted amino.

In some embodiments, provided is a compound of formula III n is an integer equal to 1 to 2.

In some embodiments, the present invention provides a compound of formula IV, a stereoisomer, tautomer, or pharmaceutically acceptable salt thereof:

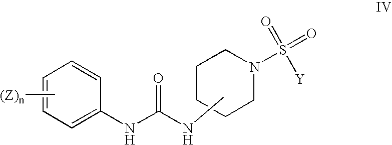

wherein:
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl; and
n is an integer equal to 1, 2, or 3;
provided that

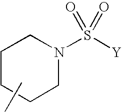

is not

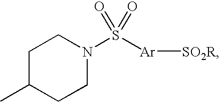

wherein Ar is arylene, substituted arylene, heteroarylene or substituted heteroarylene, R is amino or substituted amino.

In some embodiments, provided is a compound of formula IV wherein Z is independently selected from the group consisting of fluoro and fluoroalkyl, and n is an integer equal to 1 to 2.

In some embodiments, provided is a compound of formula I, II, III, or IV

wherein

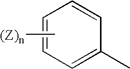

represents

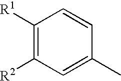

wherein R¹ and R² are independently selected from the group consisting of halogen and haloalkyl.

In some embodiments, provided is a compound of formula I, II, III, or IV wherein R¹ and R² are independently selected from the group consisting of fluorine, chlorine and trifluoromethyl.

In some embodiments, provided is a compound of formula I, II, III, or IV wherein R¹ and R² are trifluoromethyl.

In some embodiments, provided is a compound of formula I, II, III, or IV wherein R¹ and R² are fluorine.

In some embodiments, provided is a compound of formula I, II, III, or IV wherein R¹ is trifluoromethyl and R² is hydrogen.

In some embodiments, provided is a compound of formula I, II, III, or IV wherein R¹ hydrogen and R² is trifluoromethyl.

In some embodiments, provided is a compound of formula I, II, III, or IV wherein R¹ is chlorine and R² is hydrogen.

In some embodiments, provided is a compound of formula I, II, III, or IV wherein Y is selected from the group consisting of C₁-C₅ alkyl, phenyl, substituted phenyl, pyridinyl, substituted pyridinyl, imidazolyl, substituted imidazolyl, substituted imidazolyl, or imidazolylalkyl, morpholinyl, substituted morpholinyl, and morpholinylalkyl.

In some embodiments, provided is a compound of formula I, II, III, or IV wherein Y is phenyl, fluorophenyl, chlorophenyl, trifluoromethyl phenyl, or carboxyl phenyl.

In some embodiments, provided is a compound of formula I, II, III, or IV wherein Y is pyridinyl.

In some embodiments, provided is a compound of formula I, II, III, or IV wherein Y is morpholinyl, or morpholinyl-(C₁-C₃)alkyl.

In some embodiments, provided is a compound of formula I, II, III, or IV wherein Y is imidazolyl, methylimidazolyl, or imidazolyl-(C₁-C₃)alkyl.

In some embodiments, provided is a compound of formula I, II, III, or IV wherein Y is alkyl substituted with heterocycloalkyl or heteroaryl.

In some embodiments, provided is a compound of formula I, II, III, or IV wherein Y is alkyl substituted with hydroxyl or alkoxyl.

In some embodiments, provided is a compound of formula I, II, III, or IV wherein Y is phenyl substituted with carboxy, or haloalkyl.

In some embodiments, the present invention provides a compound of formula V, a stereoisomer, tautomer, or pharmaceutically acceptable salt thereof:

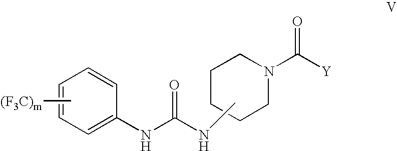

wherein:
Y is selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; and
m is an integer equal to 1, 2, or 3, preferably an integer equal to 1 or 2;
provided that

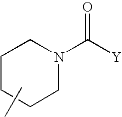

is not

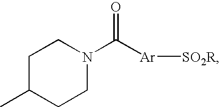

wherein Ar is arylene, substituted arylene, heteroarylene or substituted heteroarylene, and R is amino or substituted amino.

In some embodiments, the present invention provides a compound of formula VI, a stereoisomer, tautomer, or pharmaceutically acceptable salt thereof:

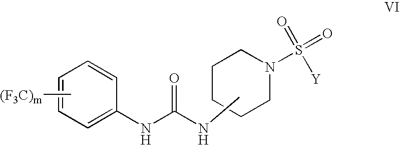

wherein:
Y is selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; and
m is an integer equal to 1, 2, or 3, preferably an integer equal to 1 or 2;
provided that

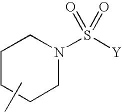

is not

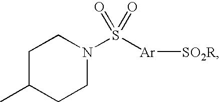

wherein Ar is arylene, substituted arylene, heteroarylene or substituted heteroarylene, R is amino or substituted amino.

In some embodiments, provided are compounds having formula VII or a stereoisomer, tautomer, or pharmaceutically acceptable salt thereof:

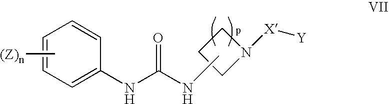

wherein:
X′ is S or SO;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3.

In some embodiments, provided are compounds having formula VIII or a stereoisomer, tautomer, or pharmaceutically acceptable salt thereof:

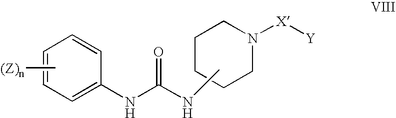

wherein:
X′ is S or SO;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3.

In some embodiments of formula VII or VIII, X′ is SO.

In some embodiments, provided is a compound of formula VII or VIII wherein

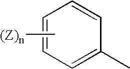

represents

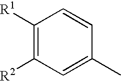

wherein R¹ and R² are independently selected from the group consisting of halogen and haloalkyl.

In some embodiments, provided is a compound of formula VII or VIII wherein R¹ and R² are independently selected from the group consisting of fluorine, chlorine and trifluoromethyl.

In some embodiments, provided is a compound of formula VII or VIII wherein R¹ and R² are trifluoromethyl.

In some embodiments, provided is a compound of formula VII or VIII wherein R¹ and R² are fluorine.

In some embodiments, provided is a compound of formula VII or VIII wherein R¹ is trifluoromethyl and R² is hydrogen.

In some embodiments, provided is a compound of formula VII or VIII wherein R¹ hydrogen and R² is trifluoromethyl.

In some embodiments, provided is a compound of formula VII or VIII wherein R¹ is chlorine and R² is hydrogen.

In some embodiments, provided is a compound of formula VII or VIII wherein Y is selected from the group consisting of C₁-C₅ alkyl, phenyl, substituted phenyl, pyridinyl, substituted pyridinyl, imidazolyl, substituted imidazolyl, substituted imidazolyl, or imidazolylalkyl, morpholinyl, substituted morpholinyl, and morpholinylalkyl.

In some embodiments, provided is a compound of formula VII or VIII wherein Y is phenyl, fluorophenyl, chlorophenyl, trifluoromethyl phenyl, or carboxyl phenyl.

In some embodiments, provided is a compound of formula VII or VIII wherein Y is pyridinyl.

In some embodiments, provided is a compound of formula VII or VIII wherein Y is morpholinyl, or morpholinyl-(C₁-C₃)alkyl.

In some embodiments, provided is a compound of formula VII or VIII wherein Y is imidazolyl, methylimidazolyl, or imidazolyl-(C₁-C₃)alkyl.

In some embodiments, provided is a compound of formula VII or VIII wherein Y is alkyl substituted with heterocycloalkyl or heteroaryl.

In some embodiments, provided is a compound of formula VII or VIII wherein Y is alkyl substituted with hydroxyl or alkoxyl.

In some embodiments, provided is a compound of formula VII or VIII wherein Y is phenyl substituted with carboxy, or haloalkyl.

In some embodiments, provided are compounds, stereoisomer, tautomer, or pharmaceutically acceptable salt thereof selected from Table 1.

TABLE 1

| No. | Structure | Name |
|---|---|---|
| 1Comparative | 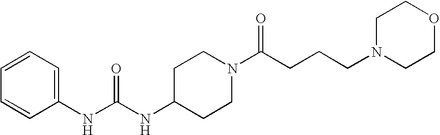 | 1-[1-(4-Morpholin-4-yl-butyryl)-piperidin-4-yl]-3-phenyl-urea |
| 2 | 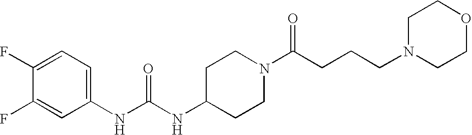 | 1-(3,4-Difluoro-phenyl)-3-[1-(4-morpholin-4-yl-butyryl)-piperidin-4-yl]-urea |
| 3 | 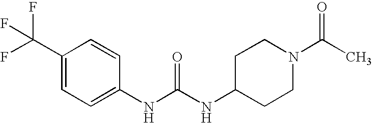 | 1-(1-Acetyl-piperidin-4-yl)-3-(4-trifluoromethyl-phenyl)-urea |
| 4 | 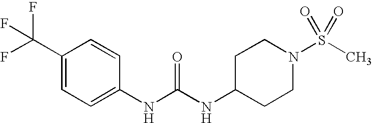 | 1-(1-Methanesulfonyl-piperidin-4-yl)-3-(4-trifluoromethyl-phenyl)-urea |
| 5Comparative | 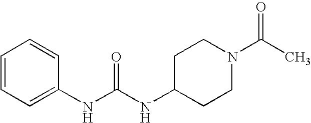 | 1-(1-Acetyl-piperidin-4-yl)-3-phenyl-urea |
| 6 | 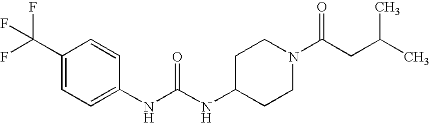 | 1-[1-(3-Methyl-butyryl)-piperidin-4-yl]-3-(4-trifluoromethyl-phenyl)-urea |
| 7 | 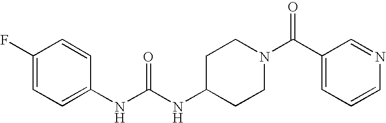 | 1-(4-Fluoro-phenyl)-3-[1-(pyridine-3-carbonyl)-piperidin-4-yl]-urea |
| 8 | 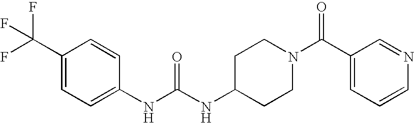 | 1-[1-(Pyridine-3-carbonyl)-piperidin-4-yl]-3-(4-trifluoromethyl-phenyl)-urea |
| 9 | 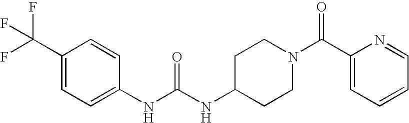 | 1-[1-(Pyridine-2-carbonyl)-piperidin-4-yl]-3-(4-trifluoromethyl-phenyl)-urea |
| 10 | 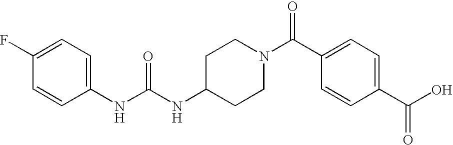 | 4-{4-[3-(4-Fluoro-phenyl)-ureido]-piperidine-1-carbonyl}-benzoic acid |
| 11 | 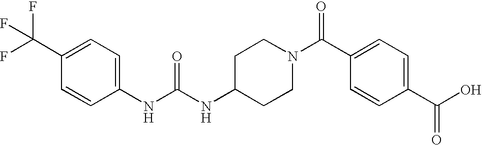 | 4-{4-[3-(4-Trifluoromethyl-phenyl)-ureido]-piperidine-1-carbonyl}-benzoic acid |
| 12 | 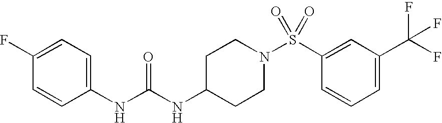 | 1-(4-Fluoro-phenyl)-3-[1-(3-trifluoromethyl-benzenesulfonyl)-piperidin-4-yl]-urea |
| 13 | 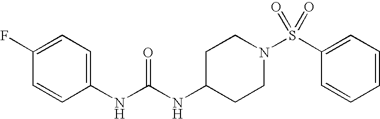 | 1-(1-Benzenesulfonyl-piperidin-4-yl)-3-(4-fluoro-phenyl)-urea |
| 14 | 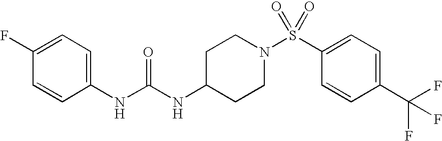 | 1-(4-Fluoro-phenyl)-3-[1-(4-trifluoromethyl-benzenesulfonyl)-piperidin-4-yl]-urea |
| 15 | 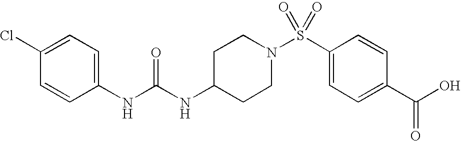 | 4-{4-[3-(4-Chloro-phenyl)-ureido]-piperidine-1-sulfonyl}-benzoic acid |
| 16 | 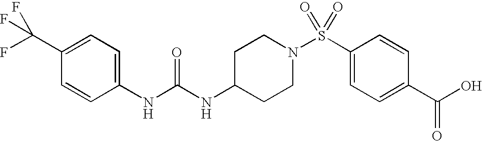 | 4-{4-[3-(4-Trifluoromethyl-phenyl)-ureido]-piperidine-1-sulfonyl}-benzoic acid |
| 17 | 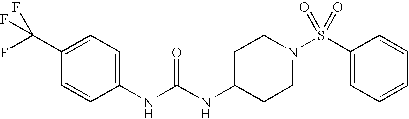 | 1-(1-Benzenesulfonyl-piperidin-4-yl)-3-(4-trifluoromethyl-phenyl)-urea |
| 18 | 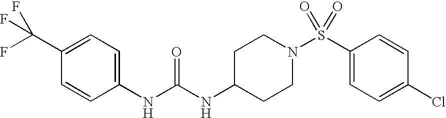 | 1-[1-(4-Chloro-benzenesulfonyl)-piperidin-4-yl]-3-(4-trifluoromethyl-phenyl)-urea |
| 19 | 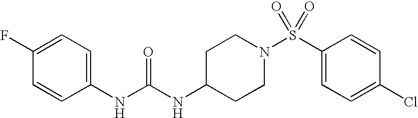 | 1-[1-(4-Chloro-benzenesulfonyl)-piperidin-4-yl]-3-(4-fluoro-phenyl)-urea |
| 20 | 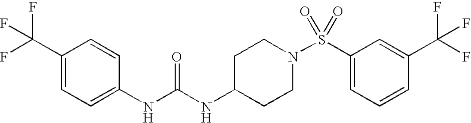 | 1-[1-(3-Trifluoromethyl-benzenesulfonyl)-piperidin-4-yl]-3-(4-trifluoromethyl-phenyl)-urea |
| 21 | 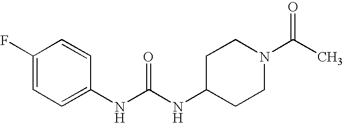 | 1-(1-Acetyl-piperidin-4-yl)-3-(4-fluoro-phenyl)-urea |
| 22 | 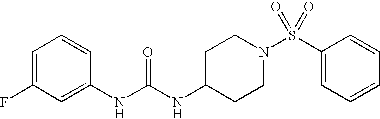 | 1-(1-Benzenesulfonyl-piperidin-4-yl)-3-(3-fluoro-phenyl)-urea |
| 23 | 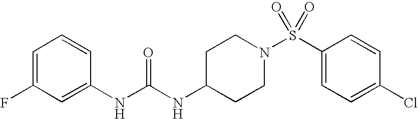 | 1-[1-(4-Chloro-benzenesulfonyl)-piperidin-4-yl]-3-(3-fluoro-phenyl)-urea |
| 24 | 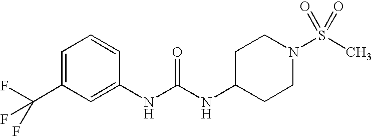 | 1-(1-Methanesulfonyl-piperidin-4-yl)-3-(3-trifluoromethyl-phenyl)-urea |
| 25 | 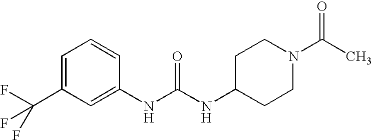 | 1-(1-Acetyl-piperidin-4-yl)-3-(3-trifluoromethyl-phenyl)-urea |
| 26 | 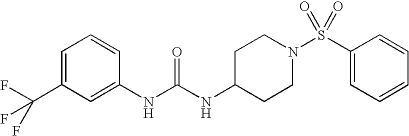 | 1-(1-Benzenesulfonyl-piperidin-4-yl)-3-(3-trifluoromethyl-phenyl)-urea |
| 27 | 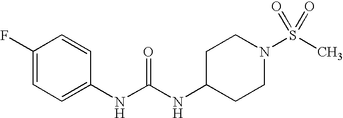 | 1-(4-Fluoro-phenyl)-3-(1-methanesulfonyl-piperidin-4-yl)-urea |
| 28 | 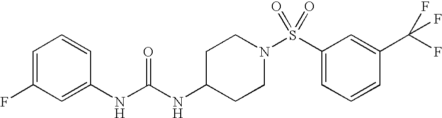 | 1-(3-Fluoro-phenyl)-3-[1-(3-trifluoromethyl-benzenesulfonyl)-piperidin-4-yl]-urea |
| 29 | 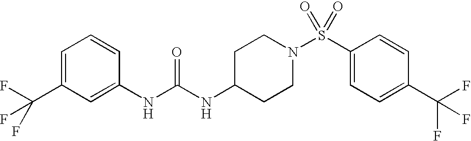 | 1-[1-(4-Trifluoromethyl-benzenesulfonyl)-piperidin-4-yl]-3-(3-trifluoromethyl-phenyl)-urea |
| 30 | 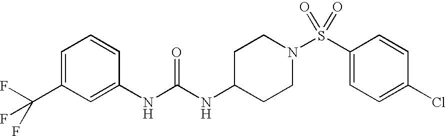 | 1-[1-(4-Chloro-benzenesulfonyl)-piperidin-4-yl]-3-(3-trifluoromethyl-phenyl)-urea |
| 31 | 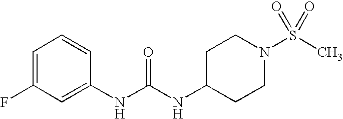 | 1-(3-Fluoro-phenyl)-3-(1-methanesulfonyl-piperidin-4-yl)-urea |
| 32 | 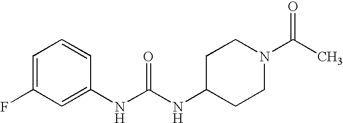 | 1-(1-Acetyl-piperidin-4-yl)-3-(3-fluoro-phenyl)-urea |
| 33 | 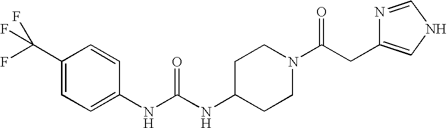 | 1-[1-(2-1H-Imidazol-4-yl-acetyl)-piperidin-4-yl]-3-(4-trifluoromethyl-phenyl)-urea |
| 34 | 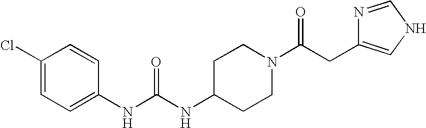 | 1-(4-Chloro-phenyl)-3-[1-(2-1H-imidazol-4-yl-acetyl)-piperidin-4-yl]-urea |
| 35 | 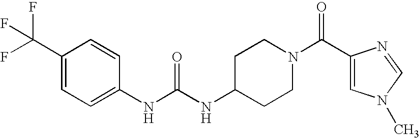 | 1-[1-(1-Methyl-1H-imidazole-4-carbonyl)-piperidin-4-yl]-3-(4-trifluoromethyl-phenyl)-urea |
| 36 | 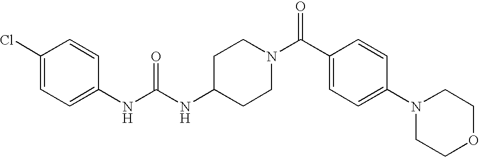 | 1-(4-Chlorophenyl)-3-(1-(4-morpholinobenzoyl)piperidin-4-yl)urea |
| 37 | 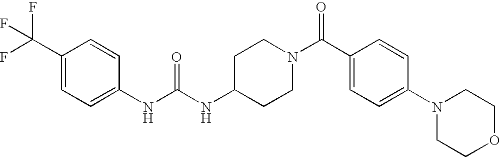 | 1-(1-(4-Morpholinobenzoyl)piperidin-4-yl)-3-(4-(trifluoromethyl)phenyl)urea |
| 38 | 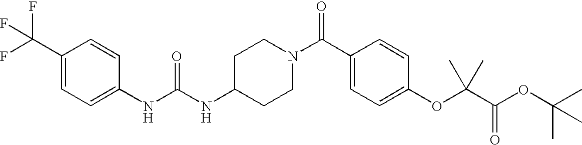 | Tert-butyl 2-methyl-2-(4-(4-(3-(4-(trifluoromethyl)phenyl)ureido)piperidine-1-carbonyl)phenoxy)propanoate |
| 39 | 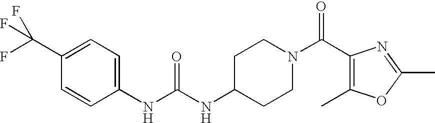 | 1-(1-(2,5-Dimethyloxazole-4-carbonyl)piperidin-4-yl)-3-(4-(trifluoromethyl)phenyl)urea |
| 40 | 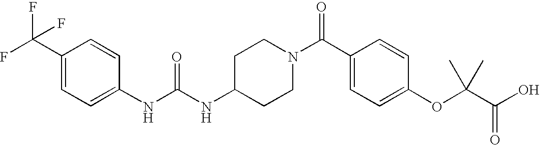 | 2-Methyl-2-(4-(4-(3-(4-(trifluoromethyl)phenyl)ureido)piperidine-1-carbonyl)phenoxy)propanoicacid |
| 41 | 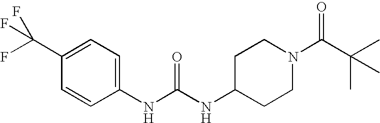 | 1-(1-Pivaloylpiperidin-4-yl)-3-(4-(trifluoromethyl)phenyl)urea |
| 42 | 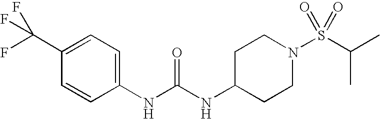 | 1-(1-(Isopropylsulfonyl)piperidin-4-yl)-3-(4-(trifluoromethyl)phenyl)urea |
| 43 | 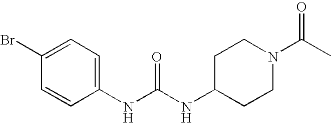 | 1-(1-acetylpiperidin-4-yl)-3-(4-bromophenyl)urea |
| 44 | 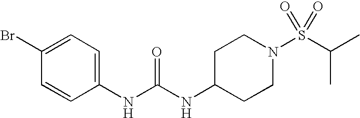 | 1-(4-bromophenyl)-3-(1-(isopropylsulfonyl)piperidin-4-yl)urea |
| 45 | 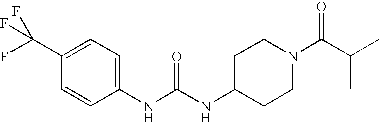 | 1-(1-isobutyrylpiperidin-4-yl)-3-(4-(trifluoromethyl)phenyl)urea |
| 46 | 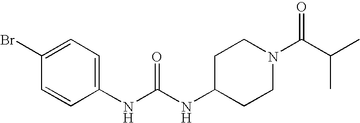 | 1-(4-bromophenyl)-3-(1-isobutyrylpiperidin-4-yl)urea |
| 47 | 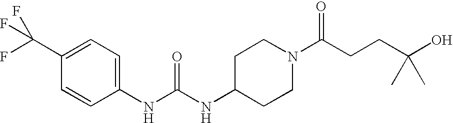 | 1-(1-(4-hydroxy-4-methylpentanoyl)piperidin-4-yl)-3-(4-(trifluoromethyl)phenyl)urea |
| 48 | 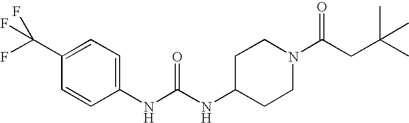 | 1-(1-(3,3-dimethylbutanoyl)piperidin-4-yl)-3-(4-(trifluoromethyl)phenyl)urea |
| 49 | 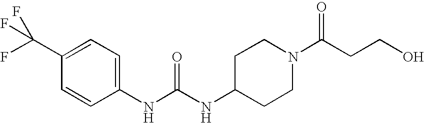 | 1-(1-(3-hydroxypropanoyl)piperidin-4-yl)-3-(4-(trifluoromethyl)phenyl)urea |
| 50 | 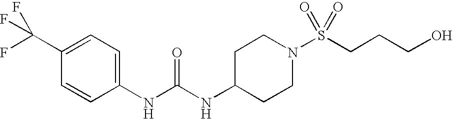 | 1-(1-(3-hydroxypropylsulfonyl)piperidin-4-yl)-3-(4-(trifluoromethyl)phenyl)urea |
| 51 | 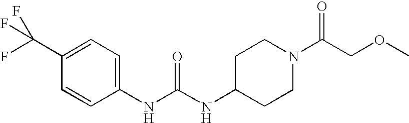 | 1-(1-(2-methoxyacetyl)piperidin-4-yl)-3-(4-(trifluoromethyl)phenyl)urea |
| 52 | 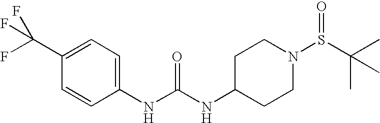 | 1-(1-(tert-butylsulfinyl)piperidin-4-yl)-3-(4-(trifluoromethyl)phenyl)urea |
| 53 | 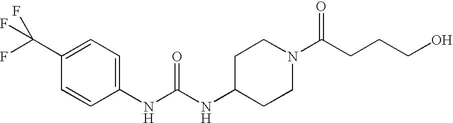 | 1-(1-(4-hydroxybutanoyl)piperidin-4-yl)-3-(4-(trifluoromethyl)phenyl)urea |
| 54 | 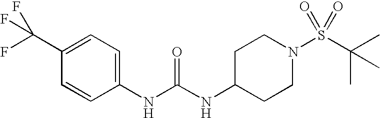 | 1-(1-(tert-butylsulfonyl)piperidin-4-yl)-3-(4-(trifluoromethyl)phenyl)urea |
| 55 | 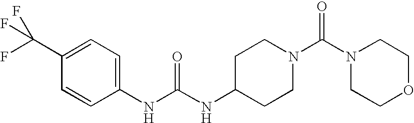 | 1-(1-(morpholine-4-carbonyl)piperidin-4-yl)-3-(4-(trifluoromethyl)phenyl)urea |

In some embodiments, provided is a pharmaceutical composition comprising a pharmaceutically acceptable carrier and a therapeutically effective amount of a compound of any one of formula I-VIII for treating a soluble epoxide hydrolase mediated disease.

In another embodiment, provided is a method for treating a soluble epoxide hydrolase mediated disease. The method comprises administering to a patient a pharmaceutical composition comprising a pharmaceutically acceptable carrier and a therapeutically effective amount of a compound of formula Ia or a stereoisomer, or pharmaceutically acceptable salt thereof:

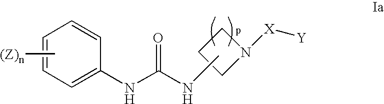

wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3.

In some aspects of the methods, the compound is of any one of formulas I-VIII.

In some aspects of the methods, the compound is any one of compounds 2-4 and 6-55 in Table 1.

It has previously been shown that inhibitors of soluble epoxide hydrolase (“sEH”) can reduce hypertension (see, e.g., U.S. Pat. No. 6,351,506). Such inhibitors can be useful in controlling the blood pressure of persons with undesirably high blood pressure, including those who suffer from diabetes.

In preferred embodiments, compounds of the invention are administered to a subject in need of treatment for hypertension, specifically renal, hepatic, or pulmonary hypertension; inflammation, specifically renal inflammation, hepatic inflammation, vascular inflammation, and lung inflammation; adult respiratory distress syndrome; diabetic complications; end stage renal disease; Raynaud syndrome; and arthritis.

### Methods to Treat ARDS and SIRS

Adult respiratory distress syndrome (ARDS) is a pulmonary disease that has a mortality rate of 50% and results from lung lesions that are caused by a variety of conditions found in trauma patients and in severe burn victims. Ingram, R. H. Jr., “Adult Respiratory Distress Syndrome,” Harrison's Principals of Internal Medicine, 13, p. 1240, 1995. With the possible exception of glucocorticoids, there have not been therapeutic agents known to be effective in preventing or ameliorating the tissue injury, such as microvascular damage, associated with acute inflammation that occurs during the early development of ARDS.

ARDS, which is defined in part by the development of alveolar edema, represents a clinical manifestation of pulmonary disease resulting from both direct and indirect lung injury. While previous studies have detailed a seemingly unrelated variety of causative agents, the initial events underlying the pathophysiology of ARDS are not well understood. ARDS was originally viewed as a single organ failure, but is now considered a component of the multisystem organ failure syndrome (MOFS). Pharmacologic intervention or prevention of the inflammatory response is presently viewed as a more promising method of controlling the disease process than improved ventilatory support techniques. See, for example, Demling, Annu. Rev. Med., 46, pp. 193-203, 1995.

Another disease (or group of diseases) involving acute inflammation is the systematic inflammatory response syndrome, or SIRS, which is the designation recently established by a group of researchers to describe related conditions resulting from, for example, sepsis, pancreatitis, multiple trauma such as injury to the brain, and tissue injury, such as laceration of the musculature, brain surgery, hemorrhagic shock, and immune-mediated organ injuries (JAMA, 268(24):3452-3455 (1992)).

The ARDS ailments are seen in a variety of patients with severe burns or sepsis. Sepsis in turn is one of the SIRS symptoms. In ARDS, there is an acute inflammatory reaction with high numbers of neutrophils that migrate into the interstitium and alveoli. If this progresses there is increased inflammation, edema, cell proliferation, and the end result is impaired ability to extract oxygen. ARDS is thus a common complication in a wide variety of diseases and trauma. The only treatment is supportive. There are an estimated 150,000 cases per year and mortality ranges from 10% to 90%.

The exact cause of ARDS is not known. However it has been hypothesized that over-activation of neutrophils leads to the release of linoleic acid in high levels via phospholipase A₂ activity. Linoleic acid in turn is converted to 9,10-epoxy-12-octadecenoate enzymatically by neutrophil cytochrome P-450 epoxygenase and/or a burst of active oxygen. This lipid epoxide, or leukotoxin, is found in high levels in burned skin and in the serum and bronchial lavage of burn patients. Furthermore, when injected into rats, mice, dogs, and other mammals it causes ARDS. The mechanism of action is not known. However, the leukotoxin diol produced by the action of the soluble epoxide hydrolase appears to be a specific inducer of the mitochondrial inner membrane permeability transition (MPT). This induction by leukotoxin diol, the diagnostic release of cytochrome c, nuclear condensation, DNA laddering, and CPP32 activation leading to cell death were all inhibited by cyclosporin A, which is diagnostic for MPT induced cell death. Actions at the mitochondrial and cell level were consistent with this mechanism of action suggesting that the inhibitors of this invention could be used therapeutically with compounds which block MPT.

Thus in one embodiment provided is a method for treating ARDS. In another embodiment, provided is a method for treating SIRS.

### Methods for Inhibiting Progression of Kidney Deterioration (Nephropathy) and Reducing Blood Pressure:

In another aspect of the invention, the compounds of the invention can reduce damage to the kidney, and especially damage to kidneys from diabetes, as measured by albuminuria. The compounds of the invention can reduce kidney deterioration (nephropathy) from diabetes even in individuals who do not have high blood pressure. The conditions of therapeutic administration are as described above.

cis-Epoxyeicosantrienoic acids (“EETs”) can be used in conjunction with the compounds of the invention to further reduce kidney damage. EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced. Without wishing to be bound by theory, it is believed that raising the level of EETs interferes with damage to kidney cells by the microvasculature changes and other pathologic effects of diabetic hyperglycemia. Therefore, raising the EET level in the kidney is believed to protect the kidney from progression from microalbuminuria to end stage renal disease.

EETs are well known in the art. EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs produced by the endothelium have anti-hypertensive properties and the EETs 11,12-EET and 14,15-EET may be endothelium-derived hyperpolarizing factors (EDHFs). Additionally, EETs such as 11,12-EET have profibrinolytic effects, anti-inflammatory actions and inhibit smooth muscle cell proliferation and migration. In the context of the present invention, these favorable properties are believed to protect the vasculature and organs during renal and cardiovascular disease states.

Inhibition of sEH activity can be effected by increasing the levels of EETs. This permits EETs to be used in conjunction with one or more sEH inhibitors to reduce nephropathy in the methods of the invention. It further permits EETs to be used in conjunction with one or more sEH inhibitors to reduce hypertension, or inflammation, or both. Thus, medicaments of EETs can be made which can be administered in conjunction with one or more sEH inhibitors, or a medicament containing one or more sEH inhibitors can optionally contain one or more EETs.

The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period in which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In preferred embodiments, the EETs, the compound of the invention, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage through the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. An exemplary coating is set forth in the Examples.

While the anti-hypertensive effects of EETs have been recognized, EETs have not been administered to treat hypertension because it was thought endogenous sEH would hydrolyse the EETs too quickly for them to have any useful effect. Surprisingly, it was found during the course of the studies underlying the present invention that exogenously administered inhibitors of sEH succeeded in inhibiting sEH sufficiently that levels of EETs could be further raised by the administration of exogenous EETs. These findings underlie the co-administration of sEH inhibitors and of EETs described above with respect to inhibiting the development and progression of nephropathy. This is an important improvement in augmenting treatment. While levels of endogenous EETs are expected to rise with the inhibition of sEH activity caused by the action of the sEH inhibitor, and therefore to result in at least some improvement in symptoms or pathology, it may not be sufficient in all cases to inhibit progression of kidney damage fully or to the extent intended. This is particularly true where the diseases or other factors have reduced the endogenous concentrations of EETs below those normally present in healthy individuals. Administration of exogenous EETs in conjunction with an sEH inhibitor is therefore expected to be beneficial and to augment the effects of the sEH inhibitor in reducing the progression of diabetic nephropathy.

The present invention can be used with regard to any and all forms of diabetes to the extent that they are associated with progressive damage to the kidney or kidney function. The chronic hyperglycemia of diabetes is associated with long-term damage, dysfunction, and failure of various organs, especially the eyes, kidneys, nerves, heart, and blood vessels. The long-term complications of diabetes include retinopathy with potential loss of vision; nephropathy leading to renal failure; peripheral neuropathy with risk of foot ulcers, amputation, and Charcot joints.

In addition, persons with metabolic syndrome are at high risk of progression to type 2 diabetes, and therefore at higher risk than average for diabetic nephropathy. It is therefore desirable to monitor such individuals for microalbuminuria, and to administer an sEH inhibitor and, optionally, one or more EETs, as an intervention to reduce the development of nephropathy. The practitioner may wait until microalbuminuria is seen before beginning the intervention. Since a person can be diagnosed with metabolic syndrome without having a blood pressure of 130/85 or higher, both persons with blood pressure of 130/85 or higher and persons with blood pressure below 130/85 can benefit from the administration of sEH inhibitors and, optionally, of one or more EETs, to slow the progression of damage to their kidneys. In some preferred embodiments, the person has metabolic syndrome and blood pressure below 130/85.

Dyslipidemia or disorders of lipid metabolism is another risk factor for heart disease. Such disorders include an increased level of LDL cholesterol, a reduced level of HDL cholesterol, and an increased level of triglycerides. An increased level of serum cholesterol, and especially of LDL cholesterol, is associated with an increased risk of heart disease. The kidneys are also damaged by such high levels. It is believed that high levels of triglycerides are associated with kidney damage. In particular, levels of cholesterol over 200 mg/dL, and especially levels over 225 mg/dL, would suggest that sEH inhibitors and, optionally, EETs, should be administered. Similarly, triglyceride levels of more than 215 mg/dL, and especially of 250 mg/dL or higher, would indicate that administration of sEH inhibitors and, optionally, of EETs, would be desirable. The administration of compounds of the present invention with or without the EETs, can reduce the need to administer statin drugs (HMG-COA reductase inhibitors) to the patients, or reduce the amount of the statins needed. In some embodiments, candidates for the methods, uses, and compositions of the invention have triglyceride levels over 215 mg/dL and blood pressure below 130/85. In some embodiments, the candidates have triglyceride levels over 250 mg/dL and blood pressure below 130/85. In some embodiments, candidates for the methods, uses and compositions of the invention have cholesterol levels over 200 mg/dL and blood pressure below 130/85. In some embodiments, the candidates have cholesterol levels over 225 mg/dL and blood pressure below 130/85.

### Methods of Inhibiting the Proliferation of Vascular Smooth Muscle Cells:

In other embodiments, compounds of any one of Formulas I-VIII and Ia or of Table 1 inhibit proliferation of vascular smooth muscle (VSM) cells without significant cell toxicity, (e.g. specific to VSM cells). Because VSM cell proliferation is an integral process in the pathophysiology of atherosclerosis, these compounds are suitable for slowing or inhibiting atherosclerosis. These compounds are useful to subjects at risk for atherosclerosis, such as individuals who have diabetes and those who have had a heart attack or a test result showing decreased blood circulation to the heart. The conditions of therapeutic administration are as described above.

The methods of the invention are particularly useful for patients who have had percutaneous intervention, such as angioplasty to reopen a narrowed artery, to reduce or to slow the narrowing of the reopened passage by restenosis. In some preferred embodiments, the artery is a coronary artery. The compounds of the invention can be placed on stents in polymeric coatings to provide a controlled localized release to reduce restenosis. Polymer compositions for implantable medical devices, such as stents, and methods for embedding agents in the polymer for controlled release, are known in the art and taught, for example, in U.S. Pat. Nos. 6,335,029; 6,322,847; 6,299,604; 6,290,722; 6,287,285; and 5,637,113. In preferred embodiments, the coating releases the inhibitor over a period of time, preferably over a period of days, weeks, or months. The particular polymer or other coating chosen is not a critical part of the present invention.

The methods of the invention are useful for slowing or inhibiting the stenosis or restenosis of natural and synthetic vascular grafts. As noted above in connection with stents, desirably, the synthetic vascular graft comprises a material which releases a compound of the invention over time to slow or inhibit VSM proliferation and the consequent stenosis of the graft. Hemodialysis grafts are a particularly preferred embodiment.

In addition to these uses, the methods of the invention can be used to slow or to inhibit stenosis or restenosis of blood vessels of persons who have had a heart attack, or whose test results indicate that they are at risk of a heart attack.

Removal of a clot such as by angioplasty or treatment with tissue plasminogen activator (tPA) can also lead to reperfusion injury, in which the resupply of blood and oxygen to hypoxic cells causes oxidative damage and triggers inflammatory events. In some embodiments, provided are methods for administering the compounds and compositions of the invention for treating reperfusion injury. In some such embodiments, the compounds and compositions are administered prior to or following angioplasty or administration of tPA.

In one group of preferred embodiments, compounds of the invention are administered to reduce proliferation of VSM cells in persons who do not have hypertension. In another group of embodiments, compounds of the invention are used to reduce proliferation of VSM cells in persons who are being treated for hypertension, but with an agent that is not an sEH inhibitor.

The compounds of the invention can be used to interfere with the proliferation of cells which exhibit inappropriate cell cycle regulation. In one important set of embodiments, the cells are cells of a cancer. The proliferation of such cells can be slowed or inhibited by contacting the cells with a compound of the invention. The determination of whether a particular compound of the invention can slow or inhibit the proliferation of cells of any particular type of cancer can be determined using assays routine in the art.

In addition to the use of the compounds of the invention, the levels of EETs can be raised by adding EETs. VSM cells contacted with both an EET and a compound of the invention exhibited slower proliferation than cells exposed to either the EET alone or to the compound of the invention alone. Accordingly, if desired, the slowing or inhibition of VSM cells of a compound of the invention can be enhanced by adding an EET along with a compound of the invention. In the case of stents or vascular grafts, for example, this can conveniently be accomplished by embedding the EET in a coating along with a compound of the invention so that both are released once the stent or graft is in position.

### Methods of Inhibiting the Progression of Obstructive Pulmonary Disease, Interstitial Lung Disease, or Asthma:

Chronic obstructive pulmonary disease, or COPD, encompasses two conditions, emphysema and chronic bronchitis, which relate to damage caused to the lung by air pollution, chronic exposure to chemicals, and tobacco smoke. Emphysema as a disease relates to damage to the alveoli of the lung, which results in loss of the separation between alveoli and a consequent reduction in the overall surface area available for gas exchange. Chronic bronchitis relates to irritation of the bronchioles, resulting in excess production of mucin, and the consequent blocking by mucin of the airways leading to the alveoli. While persons with emphysema do not necessarily have chronic bronchitis or vice versa, it is common for persons with one of the conditions to also have the other, as well as other lung disorders.

Some of the damage to the lungs due to COPD, emphysema, chronic bronchitis, and other obstructive lung disorders can be inhibited or reversed by administering inhibitors of the enzyme known as soluble epoxide hydrolase, or “sEH”. The effects of sEH inhibitors can be increased by also administering EETs. The effect is at least additive over administering the two agents separately, and may indeed be synergistic.

The studies reported herein show that EETs can be used in conjunction with sEH inhibitors to reduce damage to the lungs by tobacco smoke or, by extension, by occupational or environmental irritants. These findings indicate that the co-administration of sEH inhibitors and of EETs can be used to inhibit or slow the development or progression of COPD, emphysema, chronic bronchitis, or other chronic obstructive lung diseases which cause irritation to the lungs.

Animal models of COPD and humans with COPD have elevated levels of immunomodulatory lymphocytes and neutrophils. Neutrophils release agents that cause tissue damage and, if not regulated, will over time have a destructive effect. Without wishing to be bound by theory, it is believed that reducing levels of neutrophils reduces tissue damage contributing to obstructive lung diseases such as COPD, emphysema, and chronic bronchitis. Administration of sEH inhibitors to rats in an animal model of COPD resulted in a reduction in the number of neutrophils found in the lungs. Administration of EETs in addition to the sEH inhibitors also reduced neutrophil levels. The reduction in neutrophil levels in the presence of sEH inhibitor and EETs was greater than in the presence of the sEH inhibitor alone.

While levels of endogenous EETs are expected to rise with the inhibition of sEH activity caused by the action of the sEH inhibitor, and therefore to result in at least some improvement in symptoms or pathology, it may not be sufficient in all cases to inhibit progression of COPD or other pulmonary diseases. This is particularly true where the diseases or other factors have reduced the endogenous concentrations of EETs below those normally present in healthy individuals. Administration of exogenous EETs in conjunction with an sEH inhibitor is therefore expected to augment the effects of the sEH inhibitor in inhibiting or reducing the progression of COPD or other pulmonary diseases.

In addition to inhibiting or reducing the progression of chronic obstructive airway conditions, the invention also provides new ways of reducing the severity or progression of chronic restrictive airway diseases. While obstructive airway diseases tend to result from the destruction of the lung parenchyma, and especially of the alveoli, restrictive diseases tend to arise from the deposition of excess collagen in the parenchyma. These restrictive diseases are commonly referred to as “interstitial lung diseases”, or “ILDs”, and include conditions such as idiopathic pulmonary fibrosis. The methods, compositions, and uses of the invention are useful for reducing the severity or progression of ILDs, such as idiopathic pulmonary fibrosis. Macrophages play a significant role in stimulating interstitial cells, particularly fibroblasts, to lay down collagen. Without wishing to be bound by theory, it is believed that neutrophils are involved in activating macrophages, and that the reduction of neutrophil levels found in the studies reported herein demonstrate that the methods and uses of the invention will also be applicable to reducing the severity and progression of ILDs.

In some preferred embodiments, the ILD is idiopathic pulmonary fibrosis. In other preferred embodiments, the ILD is one associated with an occupational or environmental exposure. Exemplars of such ILDs, are asbestosis, silicosis, coal worker's pneumoconiosis, and berylliosis. Further, occupational exposure to any of a number of inorganic dusts and organic dusts is believed to be associated with mucus hypersecretion and respiratory disease, including cement dust, coke oven emissions, mica, rock dusts, cotton dust, and grain dust (for a more complete list of occupational dusts associated with these conditions, see Table 254-1 of Speizer, “Environmental Lung Diseases,” Harrison's Principles of Internal Medicine, infra, at pp. 1429-1436). In other embodiments, the ILD is sarcoidosis of the lungs. ILDs can also result from radiation in medical treatment, particularly for breast cancer, and from connective tissue or collagen diseases such as rheumatoid arthritis and systemic sclerosis. It is believed that the methods, uses and compositions of the invention can be useful in each of these interstitial lung diseases.

In another set of embodiments, the invention is used to reduce the severity or progression of asthma. Asthma typically results in mucin hypersecretion, resulting in partial airway obstruction. Additionally, irritation of the airway results in the release of mediators which result in airway obstruction. While the lymphocytes and other immunomodulatory cells recruited to the lungs in asthma may differ from those recruited as a result of COPD or an ILD, it is expected that the invention will reduce the influx of immunomodulatory cells, such as neutrophils and eosinophils, and ameliorate the extent of obstruction. Thus, it is expected that the administration of sEH inhibitors, and the administration of sEH inhibitors in combination with EETs, will be useful in reducing airway obstruction due to asthma.

In each of these diseases and conditions, it is believed that at least some of the damage to the lungs is due to agents released by neutrophils which infiltrate into the lungs. The presence of neutrophils in the airways is thus indicative of continuing damage from the disease or condition, while a reduction in the number of neutrophils is indicative of reduced damage or disease progression. Thus, a reduction in the number of neutrophils in the airways in the presence of an agent is a marker that the agent is reducing damage due to the disease or condition, and is slowing the further development of the disease or condition. The number of neutrophils present in the lungs can be determined by, for example, bronchoalveolar lavage.

### Prophylactic and Therapeutic Methods to Reduce Stroke Damage:

Inhibitors of soluble epoxide hydrolase (“sEH”) and EETs administered in conjunction with inhibitors of sEH have been shown to reduce brain damage from strokes. Based on these results, we expect that inhibitors of sEH taken prior to an ischemic stroke will reduce the area of brain damage and will likely reduce the consequent degree of impairment. The reduced area of damage should also be associated with a faster recovery from the effects of the stroke.

While the pathophysiologies of different subtypes of stroke differ, they all cause brain damage. Hemorrhagic stroke differs from ischemic stroke in that the damage is largely due to compression of tissue as blood builds up in the confined space within the skull after a blood vessel ruptures, whereas in ischemic stroke, the damage is largely due to loss of oxygen supply to tissues downstream of the blockage of a blood vessel by a clot. Ischemic strokes are divided into thrombotic strokes, in which a clot blocks a blood vessel in the brain, and embolic strokes, in which a clot formed elsewhere in the body is carried through the blood stream and blocks a vessel there. In both hemorrhagic stroke and ischemic stroke, the damage is due to the death of brain cells. Based on the results observed in our studies, we would expect at least some reduction in brain damage in all types of stroke and in all subtypes.

A number of factors are associated with an increased risk of stroke. Given the results of the studies underlying the present invention, sEH inhibitors administered to persons with any one or more of the following conditions or risk factors: high blood pressure, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, or being elderly, will reduce the area of brain damaged by a stroke. With respect to being elderly, the risk of stroke increases for every 10 years. Thus, as an individual reaches 60, 70, or 80, administration of sEH inhibitors has an increasingly larger potential benefit. As noted in the next section, the administration of EETs in combination with one or more sEH inhibitors can be beneficial in further reducing the brain damage.

In some preferred uses and methods, the sEH inhibitors and, optionally, EETs, are administered to persons who use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

Clot dissolving agents, such as tissue plasminogen activator (tPA), have been shown to reduce the extent of damage from ischemic strokes if administered in the hours shortly after a stroke. For example, tPA is approved by the FDA for use in the first three hours after a stroke. Thus, at least some of the brain damage from a stoke is not instantaneous, but rather occurs over a period of time or after a period of time has elapsed after the stroke. It is contemplated that administration of sEH inhibitors, optionally with EETs, can also reduce brain damage if administered within 6 hours after a stroke has occurred, more preferably within 5, 4, 3, or 2 hours after a stroke has occurred, with each successive shorter interval being more preferable. Even more preferably, the inhibitor or inhibitors are administered 2 hours or less or even 1 hour or less after the stroke, to maximize the reduction in brain damage. Persons of skill are well aware of how to make a diagnosis of whether or not a patient has had a stroke. Such determinations are typically made in hospital emergency rooms, following standard differential diagnosis protocols and imaging procedures.

In some preferred uses and methods, the sEH inhibitors and, optionally, EETs, are administered to persons who have had a stroke within the last 6 hours who: use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

### Metabolic Syndrome

Inhibitors of soluble epoxide hydrolase (“sEH”) and EETs administered in conjunction with inhibitors of sEH have been shown to treat one or more conditions associated with metabolic syndrome as provided for in U.S. Provisional Application Ser. No. 60/887,124 which is incorporated herein by reference in its entirety.

Metabolic syndrome is characterized by a group of metabolic risk factors present in one person. The metabolic risk factors include central obesity (excessive fat tissue in and around the abdomen), atherogenic dyslipidemia (blood fat disorders—mainly high triglycerides and low HDL cholesterol), insulin resistance or glucose intolerance, prothrombotic state (e.g., high fibrinogen or plasminogen activator inhibitor in the blood), and high blood pressure (130/85 mmHg or higher).

Metabolic syndrome, in general, can be diagnosed based on the presence of three or more of the following clinical manifestations in one subject:

a) Abdominal obesity characterized by a elevated waist circumference equal to or greater than 40 inches (102 cm) in men and equal to or greater than 35 inches (88 cm) in women;

b) Elevated triglycerides equal to or greater than 150 mg/dL;

c) Reduced levels of high-density lipoproteins of less than 40 mg/dL in women and less than 50 mg/dL in men;

d) High blood pressure equal to or greater than 130/85 mm Hg; and

e) Elevated fasting glucose equal to or greater than 100 mg/dL.

It is desirable to provide early intervention to prevent the onset of metabolic syndrome so as to avoid the medical complications brought on by this syndrome. Prevention or inhibition of metabolic syndrome refers to early intervention in subjects predisposed to, but not yet manifesting, metabolic syndrome. These subjects may have a genetic disposition associated with metabolic syndrome and/or they may have certain external acquired factors associated with metabolic syndrome, such as excess body fat, poor diet, and physical inactivity. Additionally, these subjects may exhibit one or more of the conditions associated with metabolic syndrome. These conditions can be in their incipient form.

Accordingly, one aspect, the invention provides a method for inhibiting the onset of metabolic syndrome by administering to the subject predisposed thereto an effective amount of a sEH inhibitor.

Another aspect provides a method for treating one or more conditions associated with metabolic syndrome in a subject where the conditions are selected from incipient diabetes, obesity, glucose intolerance, high blood pressure, elevated serum cholesterol, and elevated triglycerides. This method comprises administering to the subject an amount of an sEH inhibitor effective to treat the condition or conditions manifested in the subject. In one embodiment of this aspect, two or more of the noted conditions are treated by administering to the subject an effective amount of an sEH inhibitor. In this aspect, the conditions to be treated include treatment of hypertension.

sEH inhibitors are also useful in treating metabolic conditions comprising obesity, glucose intolerance, hypertension, high blood pressure, elevated levels of serum cholesterol, and elevated levels of triglycerides, or combinations thereof, regardless if the subject is manifesting, or is predisposed to, metabolic syndrome.

Accordingly, another aspect of the invention provides for methods for treating a metabolic condition in a subject, comprising administering to the subject an effective amount of a sEH inhibitor, wherein the metabolic condition is selected from the group consisting of conditions comprising obesity, glucose intolerance, high blood pressure, elevated serum cholesterol, and elevated triglycerides, and combinations thereof.

In general, levels of glucose, serum cholesterol, triglycerides, obesity, and blood pressure are well known parameters and are readily determined using methods known in the art.

Several distinct categories of glucose intolerance exist, including for example, type 1 diabetes mellitus, type 2 diabetes mellitus, gestational diabetes mellitus (GDM), impaired glucose tolerance (IGT), and impaired fasting glucose (IFG). IGT and IFG are transitional states from a state of normal glycemia to diabetes. IGT is defined as two-hour glucose levels of 140 to 199 mg per dL (7.8 to 11.0 mmol) on the 75-g oral glucose tolerance test (OGTT), and IFG is defined as fasting plasma glucose (FG) values of 100 to 125 mg per dL (5.6 to 6.9 mmol per L) in fasting patients. These glucose levels are above normal but below the level that is diagnostic for diabetes. Rao, et al., Amer. Fam. Phys. 69:1961-1968 (2004).

Current knowledge suggests that development of glucose intolerance or diabetes is initiated by insulin resistance and is worsened by the compensatory hyperinsulinemia. The progression to type 2 diabetes is influenced by genetics and environmental or acquired factors including, for example, a sedentary lifestyle and poor dietary habits that promote obesity. Patients with type 2 diabetes are usually obese, and obesity is also associated with insulin resistance.

“Incipient diabetes” refers to a state where a subject has elevated levels of glucose or, alternatively, elevated levels of glycosylated hemoglobin, but has not developed diabetes. A standard measure of the long term severity and progression of diabetes in a patient is the concentration of glycosylated proteins, typically glycosylated hemoglobin. Glycosylated proteins are formed by the spontaneous reaction of glucose with a free amino group, typically the N-terminal amino group, of a protein. HbA1c is one specific type of glycosylated hemoglobin (Hb), constituting approximately 80% of all glycosylated hemoglobin, in which the N-terminal amino group of the Hb A beta chain is glycosylated.

Formation of HbA1c irreversible and the blood level depends on both the life span of the red blood cells (average 120 days) and the blood glucose concentration. A buildup of glycosylated hemoglobin within the red cell reflects the average level of glucose to which the cell has been exposed during its life cycle. Thus the amount of glycosylated hemoglobin can be indicative of the effectiveness of therapy by monitoring long-term serum glucose regulation. The HbA1c level is proportional to average blood glucose concentration over the previous four weeks to three months. Therefore HbA1c represents the time-averaged blood glucose values, and is not subject to the wide fluctuations observed in blood glucose values, a measurement most typically taken in conjunction with clinical trials of candidate drugs for controlling diabetes.

Obesity can be monitored by measuring the weight of a subject or by measuring the Body Mass Index (BMI) of a subject. BMI is determined by dividing the subject's weight in kilograms by the square of his/her height in metres (BMI=kg/m2). Alternatively, obesity can be monitored by measuring percent body fat. Percent body fat can be measured by methods known in the art including by weighing a subject underwater, by a skinfold test, in which a pinch of skin is precisely measured to determine the thickness of the subcutaneous fat layer, or by bioelectrical impedance analysis.

### Combination Therapy

As noted above, the compounds of the present invention will, in some instances, be used in combination with other therapeutic agents to bring about a desired effect. Selection of additional agents will, in large part, depend on the desired target therapy (see, e.g., Turner, N. et al. Prog. Drug Res. (1998) 51: 33-94; Haffner, S. Diabetes Care (1998) 21: 160-178; and DeFronzo, R. et al. (eds), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed), Current Therapy In Endocrinology And Metabolism, 6th Edition (Mosby-Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). Combination therapy includes administration of a single pharmaceutical dosage formulation which contains a compound of any one of Formulas I-VIII and Ia or of Table 1 and one or more additional active agents, as well as administration of the compound and each active agent in its own separate pharmaceutical dosage formulation. For example, a compound of any one of Formulas I-VIII and Ia and one or more angiotensin receptor blockers, angiotensin converting enzyme inhibitors, calcium channel blockers, diuretics, alpha blockers, beta blockers, centrally acting agents, vasopeptidase inhibitors, renin inhibitors, endothelin receptor agonists, AGE (advanced glycation end-products) crosslink breakers, sodium/potassium ATPase inhibitors, endothelin receptor agonists, endothelin receptor antagonists, angiotensin vaccine, and the like; can be administered to the human subject together in a single oral dosage composition, such as a tablet or capsule, or each agent can be administered in separate oral dosage formulations. Where separate dosage formulations are used, the compound of any one of Formulas I-VIII and Ia and one or more additional active agents can be administered at essentially the same time (i.e., concurrently), or at separately staggered times (i.e., sequentially). Combination therapy is understood to include all these regimens.

### Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

It is contemplated that therapeutically effective amounts of the compounds will range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day. As used herein, therapeutically effective amount refers to that amount of an active compound as disclosed in embodiments of the present invention that is effective for treating or preventing the disease.

In general, compounds of this invention will be administered as pharmaceutical compositions by any one of the following routes: oral, systemic (e.g., transdermal, intranasal or by suppository), parenteral (e.g., intramuscular, intravenous or subcutaneous), or intrathecal administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the invention in combination with at least one pharmaceutically acceptable excipient. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of the compound of based on the total formulation, with the balance being one or more suitable pharmaceutical excipients. Preferably, the compound is present at a level of about 1-80 wt %. Representative pharmaceutical formulations containing a compound of any one of formulas I-VIII are described below.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

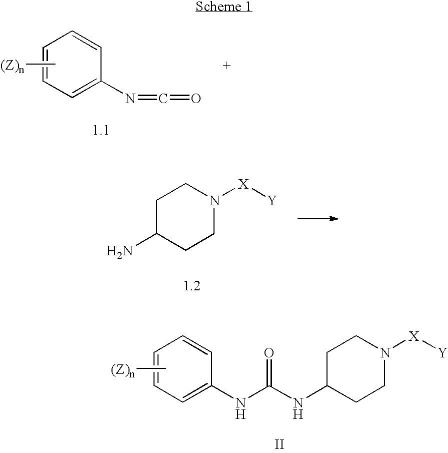

A synthesis of the compounds of the invention is shown in Scheme 1, where X, Y, Z, and n are as previously defined. For illustrative purpose only, the ring bearing —X—Y is shown as a piperidine ring. It should be pointed out that compounds of formula I wherein the X—Y bearing ring is pyrroidine or azetidine can also be similarly synthesized. Amine 1.2 reacts with the appropriate isocyanate 1.1 to form the corresponding urea of formula I. Typically, the formation of the urea is conducted using a polar solvent such as DMF (dimethylformamide) at 0 to 50° C. Compound I may be further modified using the appropriate synthetic reactions to introduce desired substituents. Such methods will be apparent to one of skill. Examples of this coupling reaction is shown in Examples 1 and 2 below.

Isocyanate 1.1 can be either known compounds or compounds that can be prepared from known compounds by conventional synthetic procedures. For example, the following isocyanate compounds are readily available: phenyl isocyanate, trifluoromethylphenyl isocyanate, chlorophenyl isocyanate and fluorophenyl isocyanate.

Amine 1.2 can be either known compounds or compounds that can be prepared from known compounds by conventional synthetic procedures. For example, amine 1.2 can be prepared according to Scheme 2, where LG represents a suitable leaving group.

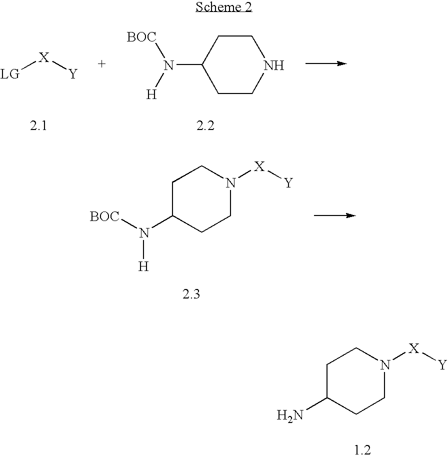

Alternatively, compounds disclosed in this invention can be synthesized as shown in Scheme 3, where PG represents a suitable protecting group. The protecting group can be any of the commonly recognized protecting groups for a secondary amine, most commonly a carbamate, such as the tert-butoxycarbonyl (Boc) group. According to Scheme 3, the appropriate isocyanate 1.1 reacts with a protected 4-aminopiperidine 3.1 to form the urea compound 3.2. Suitable conditions are then used to remove the protecting group to form amine 3.3. Reaction of amine 3.3 with LG-X—Y, where LG is a leaving group and X, Y are as previously defined, gives the desired compound of formula II. Details of this transformation can be found in Examples 3 to 42.

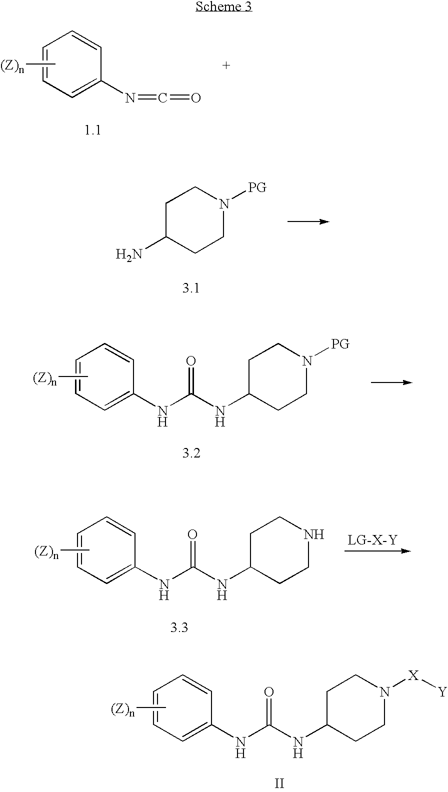

The following examples are provided to illustrate certain aspects of the present invention and to aid those of skill in the art in practicing the invention. These examples are in no way to be considered to limit the scope of the invention.

### EXAMPLES

In the examples below as well as throughout the application, the following abbreviations have the following meanings. If not defined, the terms have their generally accepted meanings.
aq.=Aqueous
bd=Broad doublet
bm=Broad multiplet
brs=Broad singlet
bt=Broad triplet
Boc=tert-Butoxycarbonyl
d=Doublet
DCM=Dichloromethane
DMAP=Dimethylaminopyridine
DMF=Dimethylformamide
DMSO=Dimethylsulfoxide
eq.=Equivalents
EtOAc=ethyl acetate
g=Gram
HPLC=high performance liquid chromatography
LCMS=Liquid chromatography mass spectroscopy
m=Multiplet
M=Molar
mg=Milligram
MHz=Megahertz
mL=Milliliter
mM=Millimolar
mmol=Millimole
m.p.=melting point
MS=Mass spectroscopy
N=Normal
NMR=nuclear magnetic resonance
s=Singlet
t=Triplet
TLC=thin layer chromatography
μL=Microliters

### Comparative Example 1

### 1-[1-(4-Morpholin-4-yl-butyryl)-piperidin-4-yl]-3-phenyl-urea (1)

### Preparation of 4-bromobutyryl chloride

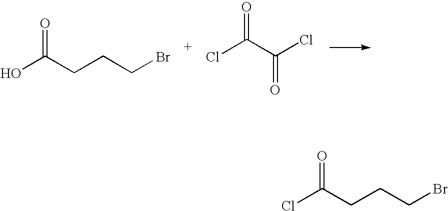

To a solution of 4-bromobutyric acid (2.00 g, 12.0 mmol, 1.2 eq.) in DCM (40 mL) was added oxalyl chloride (2 M in dichloroethane, 36 mL, 18 mmol, 1.8 eq.) and catalytic DMF (20 μL). The mixture was stirred at room temperature for 1.75 hours and the solvent was removed via rotary evaporation to provide 4-bromobutyryl chloride (high vacuum was not used to dry the product due to its potential volatility). The crude material was used immediately in the next step without further purification.

### Preparation of tert-butyl 1-(4-bromobutanoyl)piperidin-4-ylcarbamate

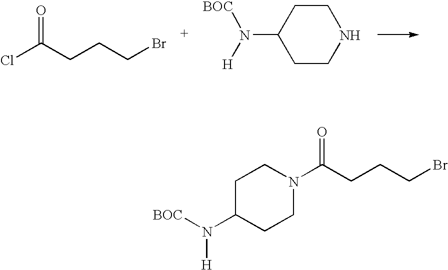

A slurry of tert-butyl piperidin-4-ylcarbamate (2.00 g, 10.0 mmol, 1.0 eq.) and pyridine (971 μL, 12.0 mmol, 1.2 eq.) in DCM (10 mL) was added over 30 seconds to a solution of crude 4-bromobutyryl chloride in DCM (20 mL). The vial containing the slurry was rinsed with DCM (10 mL) and resulting DCM wash was added to the reaction mixture. The reaction was stirred at room temperature for 2 hours and then diluted with DCM (50 mL) and washed with 5% aq. H₃PO₄ (50 mL), water (50 mL) and brine (50 mL). The organic layer was dried over Na₂SO₄, filtered, and evaporated in vacuo to provide tert-butyl 1-(4-bromobutanoyl)piperidin-4-ylcarbamate. LCMS m/z 349.4 & 351.4 [M+H]⁺. Note: During the reaction halogen exchange occurred to provide tert-butyl 1-(4-chlorobutanoyl)piperidin-4-ylcarbamate. LCMS m/z 305.4 [M+H]⁺. The crude product was used immediately in the next step without further purification.

### Preparation of tert-butyl 1-(4-morpholinobutanoyl)piperidin-4-ylcarbamate

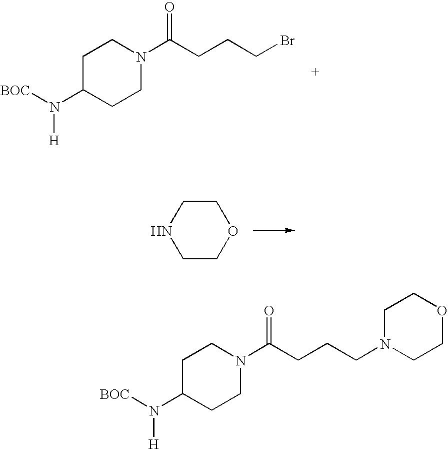

To a solution of crude tert-butyl 1-(4-bromobutanoyl)piperidin-4-ylcarbamate in DMF (40 mL) was added morpholine (4.37 mL, 50.0 mmol, 5.0 eq.). The resulting mixture was stirred at 50° C. for 14 hours. The mixture was allowed to cool, diluted with DCM (200 mL), and washed with brine (200 mL). The aqueous layer was extracted with DCM (50 mL) and the organic layers were combined and washed with brine (3×200 mL), dried over Na₂SO₄, filtered, and evaporated in vacuo to provide tert-butyl 1-(4-morpholinobutanoyl)piperidin-4-ylcarbamate (2.08 g, 5.5 mmol, 55% from tert-butyl 1-(4-bromobutanoyl)piperidin-4-ylcarbamate). LCMS m/z 356.4 [M+H]⁺. The crude product was used in the next step without further purification.

### Preparation of 1-(4-aminopiperidin-1-yl)-4-morpholinobutan-1-one

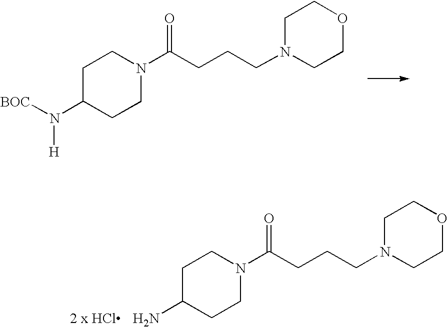

To a solution of crude tert-butyl 1-(4-morpholinobutanoyl)piperidin-4-ylcarbamate (2.08 g, 5.5 mmol, 1 eq.) in DCM (15 mL) was added HCl in dioxane (4 M, 15 mL, 60 mmol) and the mixture was stirred at room temperature for 3 hours. The solvents were removed in vacuo to provide 1-(4-aminopiperidin-1-yl)-4-morpholinobutan-1-one in quantitative yield as the dihydrochloride salt. LCMS m/z 256.4 [M+H]⁺. The crude product was used in the next step without further purification.

### Preparation of 1-[1-(4-morpholin-4-yl-butyryl)-piperidin-4-yl]-3-phenyl-urea

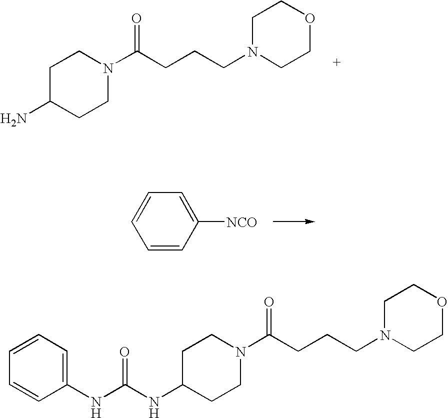

To a slurry of 1-(4-aminopiperidin-1-yl)-4-morpholinobutan-1-one (0.75 mmol) in DMF (2.5 mL) and diisopropylethylamine (0.39 mL, 2.25 mmol, 3 eq.) was added phenyl isocyanate (98 μL, 0.9 mmol, 1.2 eq.). The mixture was stirred at room temperature for 14 hours before the DMF was removed in vacuo. The residue was quenched with MeOH (ca. 1 mL), and the mixture was concentrated in vacuo. The resulting crude material was purified by reverse-phase HPLC to give 1-[1-(4-morpholin-4-yl-butyryl)-piperidin-4-yl]-3-phenyl-urea. Purity 95%; LCMS m/z 375.5 [M+H]⁺; ¹H NMR (DMSO-d₆, 300 MHz): δ 8.32 (s, 1H), 7.34 (m, 2H), 7.18 (m, 2H), 6.86 (m, 1H), 6.18 (d, J=3 Hz, 1H), 4.13-4.24 (bd, 2H), 3.6-3.85 (bm, 3H), 3.47-3.6 (m, 4H), 3.05-3.2 (bt, 1H), 2.7-2.85 (bt, 1H), 2.2-2.38 (m, 6H), 1.76-1.9 (bt, 2H), 1.58-1.70 (m, 2H), 1.1-1.4 (bm, 2H).

### Example 2

### 1-(3,4-difluoro-phenyl)-3-[1-(4-morpholin-4-yl-butyryl)-piperidin-4-yl]-urea (2)

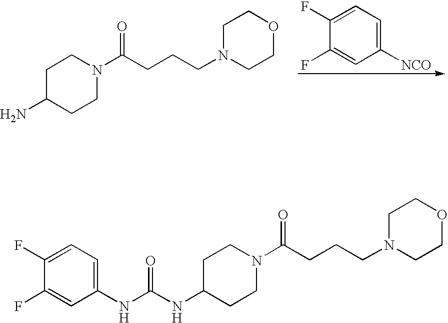

To a slurry of 1-(4-aminopiperidin-1-yl)-4-morpholinobutan-1-one (0.75 mmol, 1 eq.) in DMF (2.5 mL) and diisopropylethylamine (0.39 mL, 2.25 mmol, 3 eq.) was added 3,4-difluorophenyl isocyanate (106 μL, 0.9 mmol, 1.2 eq.). The mixture was stirred at room temperature for 14 hours before DMF was removed in vacuo. The residue was quenched with MeOH (ca. 1 mL), concentrated in vacuo, and purified by reverse-phase HPLC to give the titled compound. Purity 90%; LCMS m/z 411.5 [M+H]⁺; ¹H NMR (DMSO-d₆, 300 MHz): δ 8.62 (s, 1H), 7.58 (dd J=3 Hz, 1 Hz), 7.25 (q, J=4 Hz, 1H), 6.94-7.04 (m, 1H), 6.32 (d, J=4 Hz, 1H), 4.13-4.24 (bd 2H), 3.6-3.85 (bm, 3H), 3.47-3.6 (m, 4H), 3.04-3.22 (bt, 1H), 2.68-2.85 (bt, 1H), 2.18-2.37 (m, 6H), 1.72-1.88 (bt, 2H), 1.57-1.68 (m, 2H), 1.1-1.42 (bm, 2H).

### Example 3

### 1-(1-Acetyl-piperidin-4-yl)-3-(4-trifluoromethyl-phenyl)-urea (3)

### Preparation of tert-butyl 4-aminopiperidine-1-carboxylate

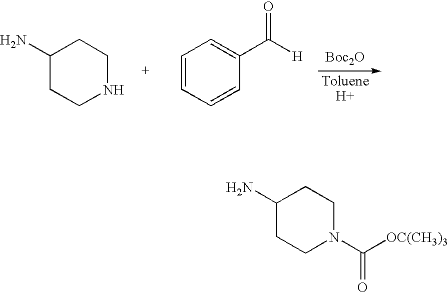

4-Aminopiperidine (5.0 g, 50 mmol, 1 eq.) was added to a solution of benzaldehyde (5.09 mL, 50 mmol, 1 eq.) in toluene (130 mL) in a 250 mL 3-necked flask fitted with a Dean-Stark trap and a condenser. A nitrogen line was connected to the top of the condenser, and the reaction was refluxed for 3 hours, during which time, water was seen to condense in the Dean-Stark trap. The reaction was cooled to room temperature and Boc anhydride (5.8 mL, 50 mmol, 1 eq.) was added over 5 minutes. The reaction was stirred over night under a blanket of N₂. The solvent was then removed under vacuum and NaHSO₄ (1M in water, 50 mL) was added to the residue. The resulting mixture was stirred vigorously for 2 hours before partitioned between diethyl ether (250 mL) and water (250 mL). The aqueous layer was separated, washed with diethyl ether (3×150 mL) and basified with NaOH solution until the pH was approximately 11. The resulting solution was extracted with DCM (4×200 mL). The combined organic layer was dried over Na₂SO₄, filtered, and evaporated to give 8.0 g of tert-butyl 4-aminopiperidine-1-carboxylate as a yellow oil.

### Preparation of tert-butyl 4-(3-(4-(trifluoromethyl)phenyl)ureido)piperidine-1-carboxylate

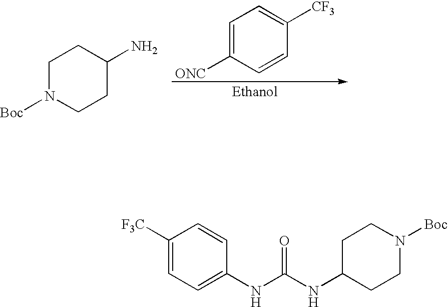

4-trifluoromethylphenyl isocyanate (1.0 eq.) was added to a solution of tert-butyl 4-aminopiperidine-1-carboxylate (1 eq.) in ethanol (10 volumes). The reaction mixture was stirred overnight at 50° C. The solvent was removed under vacuum and the crude product was crystallized in diethyl ether to give tert-butyl 4-(3-(4-(trifluoromethyl)phenyl)ureido)piperidine-1-carboxylate as a white solid.

### Preparation of 1-(piperidin-4-yl)-3-(4-(trifluoromethyl)phenyl)urea

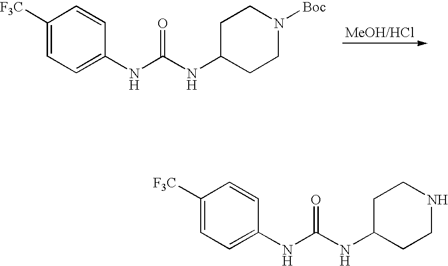

tert-butyl 4-(3-(4-(trifluoromethyl)phenyl)ureido)piperidine-1-carboxylate was stirred in MeOH/HCl overnight. The solvent was removed and the residue was stirred in diethyl ether until a white solid precipitate was seen. The precipitate was collected by filtration to give 1-(piperidin-4-yl)-3-(4-(trifluoromethyl)phenyl)urea as the hydrochloride salt.

### Preparation of 1-(1-acetyl-piperidin-4-yl)-3-(4-trifluoromethyl-phenyl)-urea

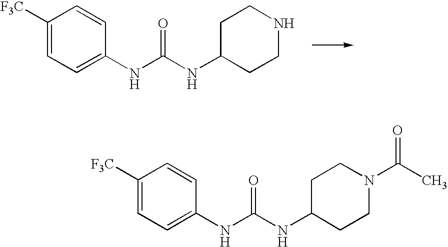

To a solution of 1-(piperidin-4-yl)-3-(4-(trifluoromethyl)phenyl)urea (10.3 g, 35.8 mmol) in DCM (150 mL) cooled with an ice water bath was added sequentially Et₃N (14.9 mL, 107 mmol) and acetic anhydride (5.0 mL, 53.8 mmol). After stirring at room temperature for 18 hours, the resulting precipitate was filtered, washed with DCM (2×50 mL), dried under a high vacuum for 4 hours to give 1-(1-acetyl-piperidin-4-yl)-3-(4-trifluoromethyl-phenyl)-urea as a white solid (8.4 g, 71%). HPLC purity 99.0%; m.p.: 240-248° C.; MS: 330 [M+H]⁺; ¹H NMR (300 MHz, DMSO-d₆) δ: 8.79 (s, 1H, NH), 7.62-7.48 (m, 4H), 6.18 (d, 1H, J=7.5 Hz, NH), 4.11 (d, J=15 Hz, 1H), 3.89-3.72 (m, 2H), 3.08 (t, 1H), 2.91 (m, 1H), 1.99 (s, 3H), 1.85-1.77 (m, 2H), 1.45-1.07 (m, 2H).

### Example 4

### 1-(1-Methanesulfonyl-piperidin-4-yl)-3-(4-trifluoromethyl-phenyl)-urea (4)

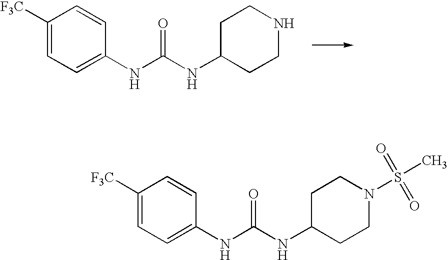

To a solution of 1-(piperidin-4-yl)-3-(4-(trifluoromethyl)phenyl)urea (10.8 g, 37.6 mmol) in DCM (150 mL) cooled with an ice water bath was added sequentially Et₃N (15.7 mL, 113 mmol) and methanesulfonyl chloride (4.37 mL, 56.4 mmol). The reaction was stirred at room temperature for 18 hours. Water (200 mL) was added and the mixture was stirred for another 18 hours. The resulting precipitate was collected by filtration, washed with water (2×50 mL), and dried for 18 hours to give the titled product (3.6 g). The supernatant from the filtration was phase separated. The organic layer was dried over Na₂SO₄, filtered, and concentrated to give an additional 4.0 g of product. The combined crude product (7.6 g) was recrystallized from EtOAc to give the pure product as a white solid (3.15 g, 23%). HPLC purity 93.8%; MS: 366 [M+H]⁺; ¹H NMR (300 MHz, CDCl₃+DMSO-d₆): δ 8.03 (s, 1H, NH), 7.12-7.00 (m, 4H), 5.86 (s, 1H), 3.37-3.20 (m, 3H), 2.95-2.82 (m, 1H), 2.58-2.41 (m, 4H), 1.72-1.58 (m, 2H), 1.24-1.08 (m, 2H).

### Comparative Example 5

### 1-(1-Acetyl-piperidin-4-yl)-3-phenyl-urea (5)

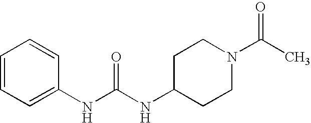

The titled compound was synthesized as an off-white solid from readily available starting materials using procedures similar to those described in the above examples. MS: 349 [M+H]⁺; ¹H NMR (300 MHz, DMSO-d₆): δ 7.2-7.5 (2d, 4H, Ar—CH), 7.0 (m, 1H, ArCH), 4.2-4.4 (d, H, CH), 3.8-4 (m, 2H, CH₂), 3.1-3.3 (tr, 1H, CH₂), 2.7-2.9 (tr, 1H, CH₂); 6.1 & 8.6 (s, 2H, NH); LCMS purity: 96.4%; yield: 70.2%.

### Example 6

### 1-[1-(3-Methyl-butyryl)-piperidin-4-yl]-3-(4-trifluoromethyl-phenyl)-urea (6)

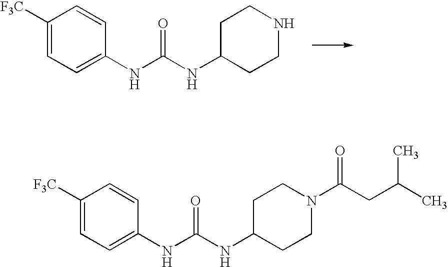

To a solution of 1-(piperidin-4-yl)-3-(4-(trifluoromethyl)phenyl)urea (9.80 g, 34.1 mmol) in DCM (150 mL) cooled with an ice water bath was added sequentially Et₃N (14.2 mL, 102 mmol) and isovaleryl chloride (6.25 mL, 51.2 mmol). The reaction was stirred at room temperature for 18 hours. Water (200 mL) was added and the mixture was stirred for another 18 hours. The resulting precipitate was collected by filtration and washed with water (2×50 mL), dried for 18 hours under a high vacuum to give the titled product as a white solid (5.4 g, 42%). HPLC purity 95.4%; MS: 372 [M+H]⁺; ¹H NMR (300 MHz, CDCl₃): δ 8.14 (S, 1H, NH), 7.32-7.18 (m, 4H), 4.24 (S, 1H, NH), 3.88-3.65 (m, 2H), 3.16-2.95 (m, 3H), 2.74-2.58 (m, 1H), 2.06-1.98 (m, 1H), 1.95-1.68 (m, 3H), 1.29-1.11 (m, 2H), 0.82-0.74 (m, 6H).

### Example 7

### 1-(4-Fluoro-phenyl)-3-[1-(pyridine-3-carbonyl)-piperidin-4-yl]-urea (7)

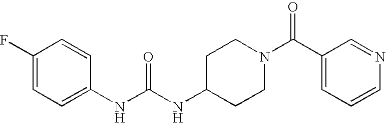

The titled compound was synthesized as an off-white solid from 1-(4-fluorophenyl)-3-(piperidin-4-yl)urea using procedures similar to those described in Example 6 above. MS: 343 [M+H]⁺; ¹HNMR (300 MHz; DMSO-d₆): δ 2.06-2.2 (m, 4H, 2CH₂), 3-4 (m, 4H, 2CH₂), 6.7 (m, 1H, CH), 7-8 (m, 6H, Ar—CH), 8-9 (br, 2H, 2NH); m.p.: 118-122° C.; LCMS purity: 98.5%; yield: 47.2%.

### Example 8

### 1-[1-(Pyridine-3-carbonyl)-piperidin-4-yl]-3-(4-trifluoromethyl-phenyl)-urea (8)

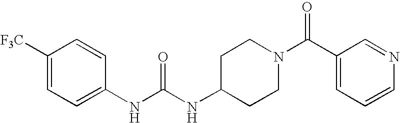

The titled compound was synthesized as an off-white solid from 1-(piperidin-4-yl)-3-(4-(trifluoromethyl)phenyl)urea using procedures similar to those described in Example 7. MS: 393 [M+H]⁺; ¹H NMR (300 MHz, DMSO-d₆) δ: 1.5-1.6 (m, 2H, CH₂), 1.8-2.2 (d, 2H, CH₂); 3.1-3.4 (m, 2H, CH₂), 4.4 (m, 1H, CH), 6.3 (d, 1H, NH), 7.3-8.3 (m, 4H, Ar—CH), 8.1 (s, 1H, Ar—CH), 8.7-8.9 (m, 3H, Ar—CH), 7.8 (brs, 1H, NH); m.p.: 213-217° C.; LCMS purity: 96%; yield: 52.8%.

### Example 9

### 1-[1-(Pyridine-2-carbonyl)-piperidin-4-yl]-3-(4-trifluoromethyl-phenyl)-urea (9)

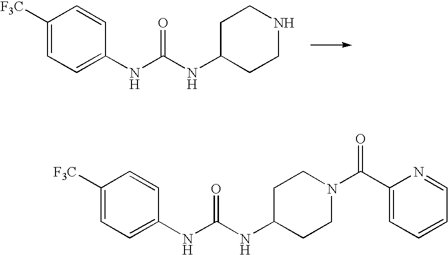

To a solution of picolinic acid (1.28 g, 10.4 mmol) in DMF (30 mL) was added sequentially Et₃N (2.9 mL, 20.9 mmol) and N-[(1H-benzotriazol-1-yl)(dimethylamino)methylene]-N-methylmethanaminium hexafluorophosphate N-oxide (HBTU) (3.29 g, 8.70 mmol) at room temperature. After 5 minutes 1-(piperidin-4-yl)-3-(4-(trifluoromethyl)phenyl)urea (2.0 g, 6.96 mmol) in DMF (20 mL) was added and the mixture was stirred for 18 hours at room temperature before being diluted with water (100 mL). The resulting precipitate was collected by filtration, washed with water (2×100 mL) and DCM (2×50 mL) and dried for 18 hours under high vacuum to give the titled compound as a white solid (2.7 g, 98%). HPLC purity 97.3%; MS: 393 [M+H]⁺, ¹H NMR (300 MHz, DMSO-d₆): δ 8.58-8.41 (m, 2H), 7.92-7.78 (m, 2H), 7.54-7.22 (m, 6H), 6.19 (s, 1H), 4.42-4.31 (m, 1H), 3.91-3.69 (m, 2H), 3.23-2.96 (m, 2H), 2.12-1.94 (m, 1H), 1.93-81 (m, 1H), 1.58-1.34 (m, 2H).

### Example 10

### 4-{4-[3-(4-Fluoro-phenyl)-ureido]-piperidine-1-carbonyl}-benzoic acid (10)

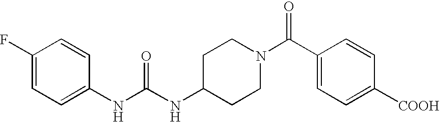

The titled compound was synthesized as an off-white solid from 1-(4-fluorophenyl)-3-(piperidin-4-yl)urea (see Example 13 below) using procedures similar to those described in Example 11 below. MS: 386 [M+H]⁺; ¹H NMR (300 MHz, CDCl₃): δ 1.5-1.6 (m, 2H, CH₂), 1.8-2.2 (d, 2H, CH₂), 3.6-3.8 (m, 2H, CH₂), 3.1-3.4 (m, 2H, CH₂), 4.4 (m, 1H, CH), 7.3-7.6 (m, 4H, Ar—CH), 8.1 (d, 2H, NH); m.p.: 195-198° C.; LCMS purity: 99.6%; yield: 52.8%.

### Example 11

### 4-{4-[3-(4-Trifluoromethyl-phenyl)-ureido]-piperidine-1-carbonyl}-benzoic acid (11)

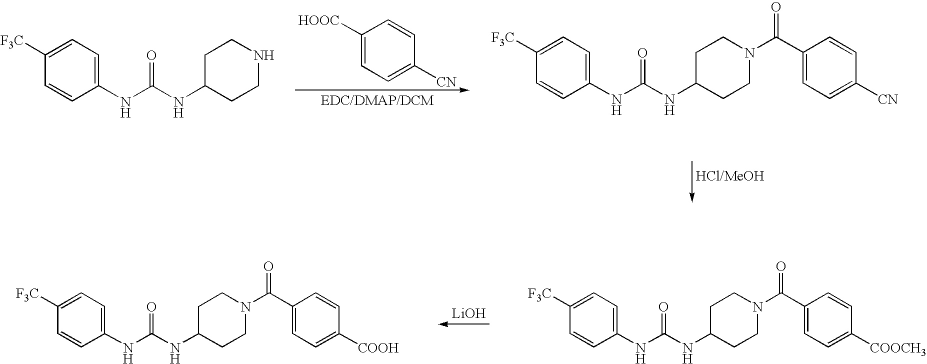

To a solution of 4-cyanobenzoic acid (1 eq.), 1-(piperidin-4-yl)-3-(4-(trifluoromethyl)-phenyl)urea (1.15 eq.), DMAP (1.1 eq.) in DCM (15 volumes) was added N-[(dimethylamino)propyl]-N′-ethylcarbodiimide hydrochloride (EDCl) (1.05 eq.) at room temperature. The reaction mixture was stirred overnight. The solvent was removed and the residue was dissolved in ethyl acetate and washed with 1N aq. NaOH, water, 1N aq. HCl and water, and dried over sodium sulfate. The crude product obtained upon removal of solvent was purified by silica gel column chromatography using EtOAc/MeOH to give 1-(1-(4-cyanobenzoyl)piperidin-4-yl)-3-(4-(trifluoromethyl)phenyl)urea.

HCl gas was bubbled in over 2 hours to a solution of 1-(1-(4-cyanobenzoyl)piperidin-4-yl)-3-(4-(trifluoromethyl)phenyl)urea in methanol at ice bath temperature. The reaction mixture was then stirred at room temperature overnight. The solvent was removed by vacuum and the residue was dissolved in ethyl acetate, washed with saturated NaHCO₃, aq. NaCl solution and water, dried over anhydrous sodium sulphate. The crude product obtained upon removal of solvent was purified by silica gel column chromatography using EtOAc/MeOH to give methyl 4-(4-(3-(4-(trifluoromethyl)phenyl)ureido)piperidine-1-carbonyl)benzoate.

Methyl 4-(4-(3-(4-(trifluoromethyl)phenyl)ureido)piperidine-1-carbonyl)benzoate (1 eq.) and LiOH (3 eq.) was added to a stirring solution of THF:MeOH:H₂O (9:1:1) at room temperature. The reaction mixture was stirred overnight. The reaction was monitored by TLC. Upon completion, the reaction mixture was concentrated under vacuum, and the residue was dissolved in H₂O and washed with ether. The aqueous layer was acidified using 1N aq. HCl solution and extracted with DCM. The organic layer was washed with brine and dried over anhydrous sodium sulfate to give the titled product.

### Example 12

### 1-(4-Fluoro-phenyl)-3-[1-(3-trifluoromethyl-benzenesulfonyl)-piperidin-4-yl]-urea (12)

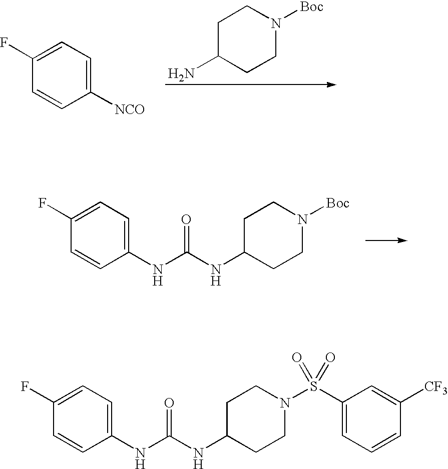

To a solution of 4-fluorophenylisocyanate (10.0 g, 72.9 mmol) in dry tetrahydrofuran (100 mL) was added tert-butyl 4-aminopiperidine-1-carboxylate (14.6 g, 72.9 mmol). The mixture was stirred at room temperature overnight. The solvent was removed and the resulting residue was washed with ether to give tert-butyl 4-(3-(4-fluorophenyl)ureido)piperidine-1-carboxylate (11.4 g, 47.6%) as a white crystalline solid. MS: 338 (M+H)⁺.

To a solution of tert-butyl 4-(3-(4-fluorophenyl)ureido)piperidine-1-carboxylate (11.4 g, 30.0 mmol) in methanol (10 mL) was added hydrochloric acid in dioxane (4M, 100 mL). The mixture was stirred at room temperature for 2 hours. The solvent was removed and the resulting solid was washed with ether and dried under vacuum. The solid (5.00 g, 17.8 mmol) was then dissolved in DCM (150 mL). m-Trifluoromethylphenylsulfonyl chloride (7.32 g, 30 mmol) was added to the solution followed by diisopropylethyl amine (4.35 g, 33.6 mmol). The resulting reaction mixture was stirred overnight. The mixture was then extracted with DCM (100 mL), washed with aqueous sodium bicarbonate solution and dried over sodium sulfate. Upon removal of solvent, the crude product was purified by column chromatography using ethyl acetate (50%) in hexane to give the product (5.00 g, 63%) as a white crystalline solid. MS: 446 (M+H)⁺; HPLC purity: 96.4%; ¹H NMR (CD₃OD, 300 MHz): δ 8.02 (m, 3H), 7.84 (t, J=7.5 Hz, 1H), 7.27 (m, 2H), 6.95 (t, J=8.7 Hz, 2H), 3.65 (m, 2H), 3.55 (m, 1H), 2.61 (t, J=11.1 Hz, 2H), 1.92 (dd, J=3 Hz, 2H), 1.48-1.61 (m, 2H).

### Example 13

### 1-(1-Benzenesulfonyl-piperidin-4-yl)-3-(4-fluoro-phenyl)-urea (13)

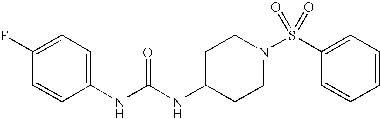

The titled compound was synthesized using procedures similar to those described in Example 12.

### Example 14

### 1-(4-Fluoro-phenyl)-3-[1-(4-trifluoromethyl-benzenesulfonyl)-piperidin-4-yl]-urea (14)

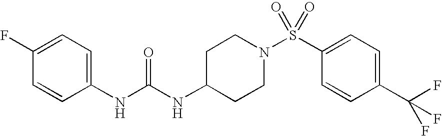

The titled compound was synthesized using procedures similar to those described in Example 12.

### Example 15

### 4-{4-[3-(4-Chloro-phenyl)-ureido]-piperidine-1-sulfonyl}-benzoic acid (15)

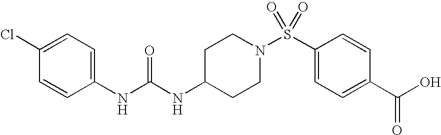

The titled compound was synthesized as a white solid from readily available starting materials using procedures similar to those described in the above examples. MS: 436 [M+H]⁺; ¹H NMR (300 MHz, DMSO-d₆) δ: 1.5-1.6 (m, 2H, CH₂); 1.8-2.0 (d, 2H, CH₂), 2.6-2.8 (m, 2H, CH₂), 3.5-3.7 (d, m, 3H, CH₂, CH), 7.2-8.3 (d, 4H, ArCH), 13.4 (brs, 1H, COOH), 8.5 & 6.3 (d, 2H, NH); m.p.: 266-270° C.; LCMS purity: 98.9%; yield: 30%.

### Example 16

### 4-{4-[3-(4-Trifluoromethyl-phenyl)-ureido]-piperidine-1-sulfonyl}-benzoic acid (16)

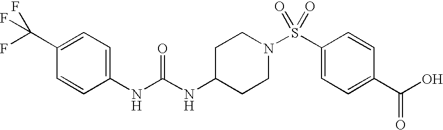

The titled compound was synthesized as a white colored solid from 1-(piperidin-4-yl)-3-(4-(trifluoromethyl)-phenyl)urea and 4-cyanobenzenesulfonyl chloride using procedures similar to those described in the above examples. MS: 472 [M+H]⁺; ¹H NMR (300 MHz, DMSO-d₆): δ 1.5-1.6 (m, 2H, CH₂), 1.8-2.0 (d, 2H, CH₂), 2.6-2.8 (m, 2H, CH₂), 3.5-3.7 (d, m, 3H, CH₂, CH), 7.8 (s, 4H, Ar—CH), 7.9-8.3 (d, 4H, ArCH—COOH), 13.6 (brs, 1H, COOH), 8.1 & 6.3 (d, 2H, NH); m.p.: 188-192° C.; LCMS purity: 99.6%; yield: 20%.

### Example 17

### 1-(1-Benzenesulfonyl-piperidin-4-yl)-3-(4-trifluoromethyl-phenyl)-urea (17)

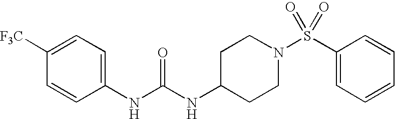

The titled compound was synthesized as a white solid from 1-(piperidin-4-yl)-3-(4-(trifluoromethyl)phenyl)urea by procedures similar to those described in Example 4. MS 428 [M+H]⁺, ¹H NMR (300 MHz, CDCl₃): δ 1.5-1.6 (m, 2H, CH₂), 1.8-2.0 (d, 2H, CH₂), 2.6-2.8 (m, 2H, CH₂), 3.5-3.7 (d, m, 3H, CH₂, CH), 7.6-7.8 (m, 9H, Ar—CH), 8.1 & 6.3 (d, 2H, NH); m.p.: 248-252° C.; LCMS purity: 99.4%; yield: 70%.

### Example 18

### 1-[1-(4-Chloro-benzenesulfonyl)-piperidin-4-yl]-3-(4-trifluoromethyl-phenyl)-urea (18)

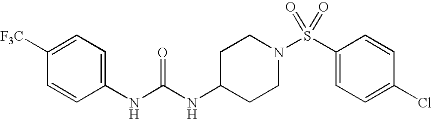

The titled compound was synthesized as a white solid from 1-(piperidin-4-yl)-3-(4-(trifluoromethyl)phenyl)urea using procedures similar to those described in Example 4. MS: 462 [M+H]⁺; ¹H NMR (300 MHz, DMSO-d₆): δ 1.5-1.6 (m, 2H, CH₂), 1.8-2.2 (d, 2H, CH₂), 3.6-3.8 (m, 2H, CH₂), 3.1-3.4 (d, m, 3H, CH₂, CH), 7.6-7.8 (m, 4H, Ar—CH), 8.1 & 6.3 (d, 2H, NH); m.p.: 266-270° C.; LCMS purity: 99.0%; yield: 60%.

### Example 19

### 1-[1-(4-Chloro-benzenesulfonyl)-piperidin-4-yl]-3-(4-fluoro-phenyl)-urea (19)

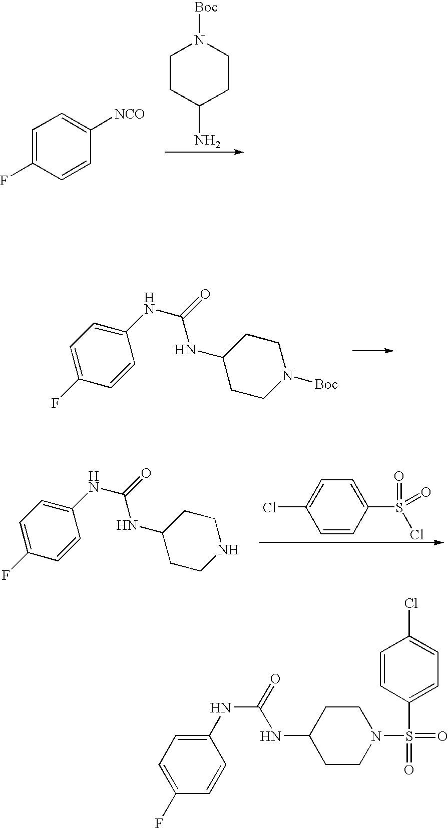

To a solution of tert-butyl 4-aminopiperidine-1-carboxylate (3.8 g, 19 mmol) in dry DMF (20 mL) was added 4-fluorophenyl isocyanate (2.6 g, 19 mmol) at 0° C. The mixture was stirred for 8 hours at 0-10° C. After completion of the reaction, the reaction mixture was poured into water and extracted with ethyl acetate, washed with water and brine, dried over anhydrous sodium sulfate and evaporated under reduced pressure to give 4.45 g of crude tert-butyl 4-(3-(4-fluorophenyl)ureido)piperidine-1-carboxylate.

The crude tert-butyl 4-(3-(4-fluorophenyl)ureido)piperidine-1-carboxylate was then added to 80 mL of 4N HCl/dioxane and allowed to stir at room temperature for 4 hours. The reaction mixture was next poured into 100 mL of water, neutralized with 1N NaOH, and extracted with dichloromethane. The organic layer was then washed with saturated sodium bicarbonate solution followed by brine and distilled under reduced pressure to give 3.2 g of the crude material. The crude material was then purified by flash column chromatography on silica gel (120-240 mesh) with ethyl acetate:hexane (2:3) as eluent (R_{f}=0.45) to give 2.8 g of 1-(4-fluorophenyl)-3-(piperidin-4-yl)urea.

1-(4-fluorophenyl)-3-(piperidin-4-yl)urea (100 mg, 4.21 mmol) was dissolved in 20 mL of dry DCM followed by addition of triethyl amine (43 mg, 0.422 mmol). The mixture was allowed to stir at room temperature for 10 minutes. 4-Chlorobenzene sulfonyl chloride (88 mg, 0.419 mmol) was then added slowly and the reaction mixture was allowed to stir at room temperature for another 1 hour. After completion of the reaction, the mixture was poured into 50 mL of water and extracted with DCM, washed with saturated sodium bicarbonate solution and brine. The residue obtained upon removal of solvent was purified by flash column chromatography on silica gel (120-240 mesh) with ethyl acetate:hexane (1:4) as eluent (R_{f}=0.58) to give 125 mg of 1-[1-(4-chloro-benzenesulfonyl)-piperidin-4-yl]-3-(4-fluoro-phenyl)-urea as a white solid. ¹H NMR (200 MHz, CDCl₃): δ 7.82 (m, 2H), 7.62-7.60 (m, 2H), 7.50-7.42 (m, 2H), 6.95 (m, 2H), 5.90 (brs, 2H), 3.60 (m, 1H), 2.80-2.78 (m, 4H), 1.82-1.70 (m, 4H); MS: 412 (M+H)⁺; m.p.: 160-162° C.

### Example 20

### 1-[1-(3-Trifluoromethyl-benzenesulfonyl)-piperidin-4-yl]-3-(4-trifluoromethyl-phenyl)-urea (20)

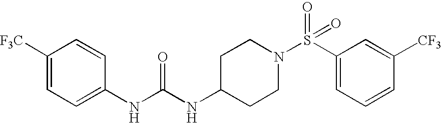

The titled compound was synthesized as a pale brown solid from readily available starting materials using procedures similar to those described in the above examples. MS: 496 [M+H]⁺; ¹H NMR (300 MHz, DMSO-d₆): δ 1.5-1.6 (m, 2H, CH₂), 1.8-2.0 (d, 2H, CH₂), 2.6-2.8 (m, 2H, CH₂), 3.5-3.7 (m, 3H, CH₂, CH), 7.6 (brs, 4H, ArH), 7.8-8.3 (d, 4H, ArCH), 13.4 (brs, 1H, COOH), 8.8 & 6.3 (brs, d, 2H, NH); m.p.: 246-251° C.; LCMS purity: 98.89%; yield: 70%.

### Example 21

### 1-(1-Acetyl-piperidin-4-yl)-3-(4-fluoro-phenyl)-urea (21)

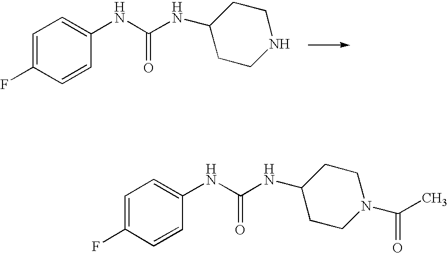

To a solution of 1-(4-fluorophenyl)-3-(piperidin-4-yl)urea (100 mg, 0.421 mmol) in 20 mL of dry dichloromethane was added triethyl amine (43 mg, 0.422 mmol). The mixture was stirred at room temperature for 10 minutes. Freshly distilled acetyl chloride (33 mg, 0.419 mmol) was then added slowly and the reaction mixture was allowed to stir at room temperature for another 2 hours. The reaction was then poured into 50 mL of water and extracted with DCM, washed with saturated sodium bicarbonate solution and brine. The crude product obtained upon removal of solvent was purified by flash column chromatography on silica gel (120-240 mesh) with ethyl acetate:hexane (1:4) as eluent (R_{f}=0.65) to give 95 mg of 1-(1-acetyl-piperidin-4-yl)-3-(4-fluoro-phenyl)-urea as a white solid. ¹H NMR (200 MHz, CDCl₃): δ 7.50-7.42 (m, 2H), 6.95-6.85 (m, 2H), 5.80 (brs, 2H), 3.60 (m, 1H), 2.80-2.78 (m, 4H), 2.0 (s, 1H), 1.82-1.70 (m, 4H); MS: 280 (M+H)⁺; m.p.: 146-148° C.

### Example 22

### 1-(1-Benzenesulfonyl-piperidin-4-yl)-3-(3-fluoro-phenyl)-urea (22)

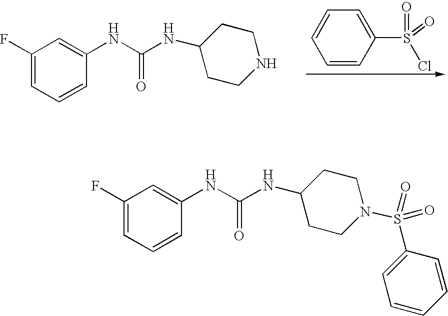

To a solution of 1-(3-fluorophenyl)-3-(piperidin-4-yl)urea (see Example 24 below, 100 mg, 4.19 mmol) in 20 mL of dry DCM was added triethylamine (43 mg, 0.422 mmol) and the mixture was stirred at 0° C. for 10 minutes. Freshly distilled benzenesulfonyl chloride (80 mg, 0.456 mmol) was then added slowly and the reaction was stirred at room temperature for another 2 hours. After completion of the reaction, the mixture was poured into 50 mL of water and extracted with dichloromethane. The organic extract was washed with saturated sodium bicarbonate solution, brined, and evaporated. The residue was purified by flash column chromatography on silica gel (120-240 mesh) with ethyl acetate:hexane (1:4) as eluent (R_{f}=0.65) to give 95 mg of 1-(1-benzenesulfonyl-piperidin-4-yl)-3-(3-fluoro-phenyl)-urea as a white solid. ¹H NMR (200 MHz, CDCl₃): δ 7.75 (s, 1H), 7.50-7.22 (m, 7H), 6.95-6.85 (m, 2H), 6.80 (brs, 2H), 3.60 (m, 1H), 2.80-2.78 (m, 4H), 1.82-1.70 (m, 4H); MS 378 (M+H)⁺; m.p.: 186-188° C.

### Example 23

### 1-[1-(4-Chloro-benzenesulfonyl)-piperidin-4-yl]-3-(3-fluoro-phenyl)-urea (23)

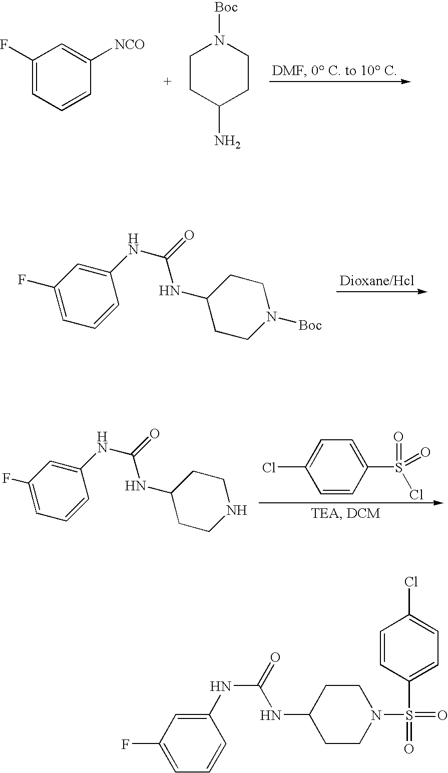

To a solution of tert-butyl 4-aminopiperidine-1-carboxylate (1.5 g, 7.5 mmol) in dry DMF (20 mL) was added 3-fluorophenyl isocyanate (1.02 g, 7.5 mmol) at 0° C. The resulting mixture was stirred for 2 hours at 0-10° C. After completion of the reaction, the reaction mixture was poured into water, extracted with ethyl acetate, washed with water and brine, dried over anhydrous sodium sulfate and evaporated under reduced pressure to give 2.45 g of crude tert-butyl 4-(3-(3-fluorophenyl)ureido)piperidine-1-carboxylate. The crude product was then dissolved in 50 ml of 4N HCl/dioxane and allowed to stir at room temperature for 4 hours. The reaction mixture was then poured into 100 mL of water, neutralized by 1N NaOH and extracted with DCM. The organic layer was then washed with saturated sodium bicarbonate solution followed by brine. The crude product obtained upon removal of solvent was purified by flash column chromatography on silica gel (120-240 mesh) with ethyl acetate:hexane (2:3) as eluent (R_{f}=0.45) to give 1.28 g of pure 1-(3-fluorophenyl)-3-(piperidin-4-yl)urea.

100 mg (4.19 mmol) of 1-(3-fluorophenyl)-3-(piperidin-4-yl)urea was dissolved in 20 mL of dry DCM followed by addition of triethylamine (43 mg, 0.422 mmol). The mixture was allowed to stir at room temperature for 10 minutes. 4-Chlorobenzenesulfonyl chloride (88 mg, 0.419 mmol) was then added slowly and the mixture was stirred at room temperature for another 1 hour. After completion of the reaction, the mixture was poured into 50 mL of water and extracted with DCM, washed with saturated sodium bicarbonate solution and brine, concentrated and purified by flash column chromatography on silica gel (120-240 mesh) with ethyl acetate:hexane (1:4) as eluent (R_{f}=0.58) to give 85 mg of pure 1-[1-(4-chloro-benzenesulfonyl)-piperidin-4-yl]-3-(3-fluoro-phenyl)-urea as a white solid. ¹H NMR (200 MHz, CDCl₃): δ 7.82 (s, 1H), 7.62-7.60 (m, 2H), 7.50-7.42 (m, 3H), 6.95 (m, 2H), 5.90 (brs, 2H), 3.60 (m, 1H), 2.80-2.78 (m, 4H), 1.82-1.70 (m, 4H); MS: 412 (M+H)⁺; m.p.: 245-248° C.

### Example 24

### 1-(1-Methanesulfonyl-piperidin-4-yl)-3-(3-trifluoromethyl-phenyl)-urea (24)

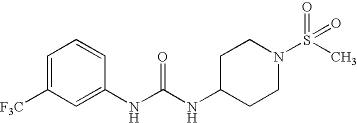

The titled compound was synthesized as an off-white solid using procedures similar to those described in Example 27 below. MS: 366 [M+H]⁺; ¹H NMR (300 MHz, DMSO-d₆): δ 1.5-1.6 (m, 2H, CH₂), 1.8-2.2 (d, 2H, CH₂), 3.1-3.4 (m, 2H, CH₂), 4.4 (m, 1H, CH), 3.0 (s, 3H, CH₃), 6.3 (d, 1H, NH), 7.3-7.5 (m, 3H, Ar—CH), 8.9 (s, 1H, Ar—CH), 7.8 (brs, 1H, NH); 25. m.p.: 226-229° C.; LCMS purity: 96.9%; yield: 55.5%.

### Example 25

### 1-(1-Acetyl-piperidin-4-yl)-3-(3-trifluoromethyl-phenyl)-urea (25)

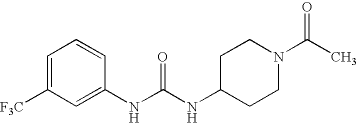

The titled compound was synthesized as a white solid using procedures similar to those described in Example 27 below. MS: 330 [M+H]⁺; ¹H NMR (300 MHz, DMSO-d₆): δ 1.2-1.5 (m, 2H, CH₂), 1.8-2.2 (d, 2H, CH₂), 2.9-3.2 (t, 2H, CH₂), 3.8 (m, 2H, CH₂), 4.4 (m, 1H, CH), 2.0 (s, 3H, CH₃), 6.3 (d, 1H, NH), 7.3-7.5 (m, 3H, Ar—CH), 8.9 (s, 1H, Ar—CH), 7.8 (brs, 1H, NH); m.p.: 212-217° C.; LCMS purity: 96.9%; yield: 55.5%.

### Example 26

### 1-(1-Benzenesulfonyl-piperidin-4-yl)-3-(3-trifluoromethyl-phenyl)-urea (26)

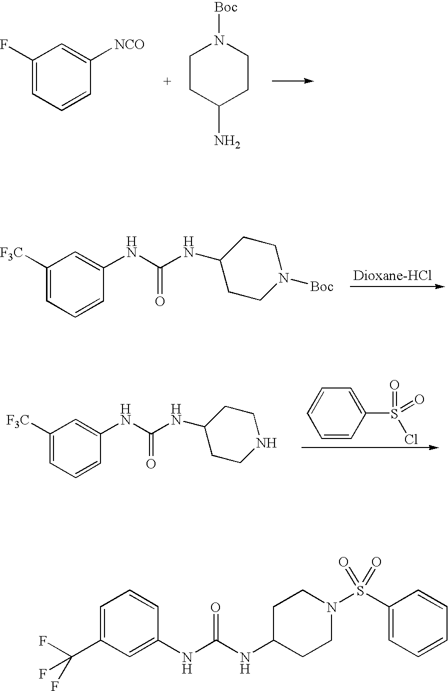

To a solution of tert-butyl 4-aminopiperidine-1-carboxylate (1.5 g, 7.4 mmol) in dry DMF (20 mL) was added 3-trifluoromethylphenyl isocyanate (1.4 g, 7.4 mmol) at 0° C. The mixture was stirred for 5 hours at 0-10° C. After completion of the reaction, the reaction mixture was poured into water and extracted with ethyl acetate. The organic extract was washed with water and brine, dried over anhydrous sodium sulfate and evaporated under reduced pressure to give 2.85 g of crude product. The crude product was dissolved in 80 mL of 4N HCl/dioxane and was stirred at room temperature for 4 hours. The reaction mixture was then poured into 100 mL of water, neutralized with 1N NaOH and extracted with dichloromethane. The organic layer was washed with saturated sodium bicarbonate solution and brine, and evaporated. The resulting crude product was purified by flash column chromatography on silica gel (120-240 mesh) with ethyl acetate/hexane (2:3) as eluent (R_{f}=0.45) to give 1-(piperidin-4-yl)-3-(3-(trifluoromethyl)phenyl)urea.

200 mg (0.696 mmol) of 1-(piperidin-4-yl)-3-(3-(trifluoromethyl)phenyl)urea was dissolved in 20 mL of dry dichloromethane followed by addition of triethylamine (118 mg, 1.16 mmol). The resulting mixture was stirred at room temperature for 10 minutes. Benzenesulfonyl chloride (136 mg, 0.773 mmol) was then added slowly and the reaction mixture was allowed to stir at room temperature for another 1 hour. After completion of the reaction, the mixture was poured into 50 mL of water, extracted with dichloromethane, washed with saturated sodium bicarbonate solution and brine. The residue obtained upon removal of solvent was purified by flash column chromatography on silica gel (120-240 mesh) with ethyl acetate/hexane (1:4) (R_{f}=0.68) to give 115 mg of 1-(1-benzenesulfonyl-piperidin-4-yl)-3-(3-trifluoromethyl-phenyl)-urea as a white solid. ¹H NMR (200 MHz, CDCl₃): δ 7.82 (m, 2H), 7.62-7.70 (s, 1H); 7.50-7.42 (m, 6H), 6.95 (m, 2H), 5.90 (brs, 2H), 3.60 (m, 1H), 2.80-2.78 (m, 4H), 1.82-1.70 (m, 4H); MS: 428 (M+H)⁺.

### Example 27

### 1-(4-Fluoro-phenyl)-3-(1-methanesulfonyl-piperidin-4-yl)-urea (27)

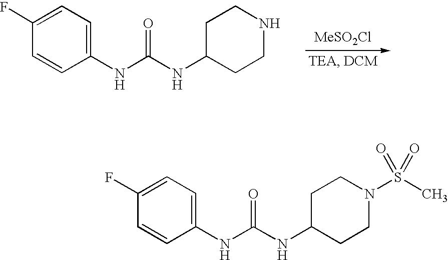

To a solution of 1-(4-fluorophenyl)-3-(piperidin-4-yl)urea (100 mg, 0.419 mmol) in 20 mL of dry dichloromethane was added triethylamine (43 mg, 0.422 mmol). The mixture was stirred at room temperature for 10 minutes. Freshly distilled methanesulfonyl chloride (49 mg, 0.419 mmol) was then added slowly and the reaction mixture was allowed to stir at room temperature for another 2 hours. After completion of the reaction, the mixture was poured into 50 mL of water, extracted with dichloromethane, washed with saturated sodium bicarbonate solution and brine, and evaporated. The residue was purified by flash column chromatography on silica gel (120-240 mesh) with ethyl acetate/hexane (1:4) as eluent (R_{f}=0.65) to give 85 mg of 1-(4-fluoro-phenyl)-3-(1-methanesulfonyl-piperidin-4-yl)-urea as a white solid. ¹H NMR (200 MHz, CDCl₃): δ 7.50-7.42 (m, 2H), 6.95-6.85 (m, 2H), 5.80 (bs, 2H), 3.60 (m, 1H), 3.12 (s, 1H), 2.80-2.78 (m, 4H), 1.82-1.70 (m, 4H); MS: 316 (M+H)⁺; m.p.: 186-188° C.

### Example 28

### 1-(3-Fluoro-phenyl)-3-[1-(3-trifluoromethyl-benzenesulfonyl)-piperidin-4-yl]-urea (28)

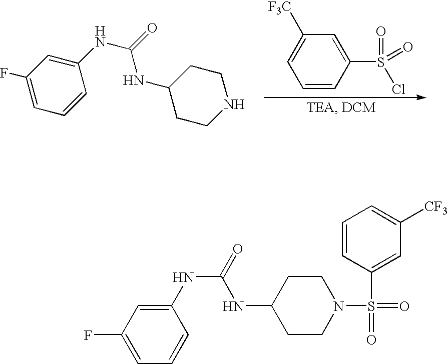

To a solution of 1-(3-fluorophenyl)-3-(piperidin-4-yl)urea (100 mg, 0.421 mmol) in 20 mL of dry dichloromethane was added triethylamine (43 mg, 0.422 mmol). The mixture was allowed to stir at 0° C. for 10 minutes. 3-Trifluoromethyl-benzenesulfonyl chloride (113 mg, 0.463 mmol) was then added slowly and the reaction was stirred at room temperature for another 2 hours. After completion of the reaction, the mixture was poured into 50 mL of water and extracted with dichloromethane. The organic extract was washed with saturated sodium bicarbonate solution, brined, and evaporated. The residue was purified by flash column chromatography on silica gel (120-240 mesh) with ethyl acetate:hexane (1:4) as the eluent (R_{f}=0.65) to give 155 mg of 1-(3-fluoro-phenyl)-3-[1-(3-trifluoromethyl-benzenesulfonyl)-piperidin-4-yl]-urea as a white solid. ¹H NMR (200 MHz, CDCl₃): δ 7.65 (s, 1H), 7.50-7.22 (m, 5H), 6.95-6.85 (m, 2H), 6.80 (brs, 2H), 3.60 (m, 1H), 2.80-2.78 (m, 4H), 1.82-1.70 (m, 4H); MS: 446 (M+H)⁺; m.p.: 86-88° C.

### Example 29

### 1-[1-(4-Trifluoromethyl-benzenesulfonyl)-piperidin-4-yl]-3-(3-trifluoromethyl-phenyl)-urea (29)

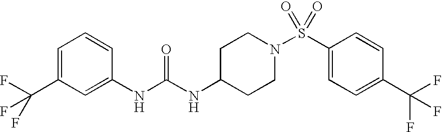

The titled compound was synthesized using procedures similar to those described in the above examples.

### Example 30

### 1-[1-(4-Chloro-benzenesulfonyl)-piperidin-4-yl]-3-(3-trifluoromethyl-phenyl)-urea (30)

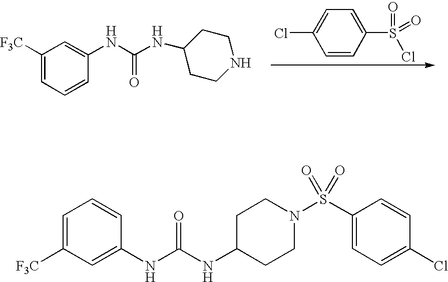

Triethylamine (132 mg, 1.30 mmol) was added to a solution of 1-(piperidin-4-yl)-3-(3-(trifluoromethyl)phenyl)urea (200 mg, 0.696 mmol) in 20 mL of dry dichloromethane. The mixture was stirred at 0° C. for 10 minutes. Freshly distilled 4-chlorobenzenesulfonyl chloride (102 mg, 0.873 mmol) was then added slowly and the resulting mixture was stirred at room temperature for another 2 hours. After completion of the reaction, the reaction mixture was poured into 50 mL of water, extracted with dichloromethane, and washed with saturated sodium bicarbonate solution and brine. The residue obtained upon removal of solvent was purified by flash column chromatography on silica gel (120-240 mesh) with ethyl acetate/hexane (1:4) as eluent (R_{f}=0.55) to give 115 mg of 1-[1-(4-chloro-benzenesulfonyl)-piperidin-4-yl]-3-(3-trifluoromethyl-phenyl)-urea as a white solid. ¹H NMR (200 MHz, CDCl₃): δ 7.55 (s, 1H), 7.50-7.22 (m, 5H), 6.90-6.85 (m, 2H), 6.80 (brs, 2H), 3.60 (m, 1H), 2.80-2.78 (m, 4H), 1.82-1.70 (m, 4H); MS: 462 (M+H)⁺; m.p.: 116-118° C.

### Example 31

### 1-(3-Fluoro-phenyl)-3-(1-methanesulfonyl-piperidin-4-yl)-urea (31)

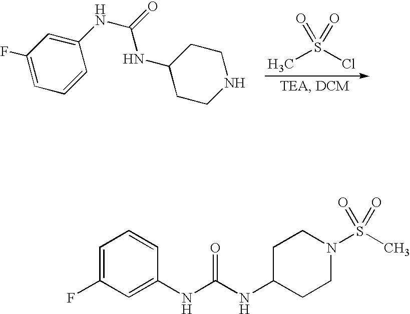

To a solution of 1-(3-fluorophenyl)-3-(piperidin-4-yl)urea (100 mg, 0.421 mmol) in 20 mL of dry dichloromethane was added triethylamine (43 mg, 0.422 mmol). The mixture was allowed to stir at 0° C. for 10 minutes. Methanesulfonyl chloride (54 mg, 0.463 mmol) was then added slowly and the resulting mixture was allowed to stir at room temperature for another 2 hours. After completion of the reaction, the mixture was poured into 50 mL of water, extracted with dichloromethane, and washed with saturated sodium bicarbonate solution and brine. The residue obtained upon removal of solvent was purified by flash column chromatography on silica gel (120-240 mesh) with ethyl acetate:hexane (1:4) as the eluent (R_{f}=0.55) to give 155 mg of 1-(3-fluoro-phenyl)-3-(1-methanesulfonyl-piperidin-4-yl)-urea as a white solid. ¹H NMR (200 MHz, CDCl₃): δ 7.65 (s, 1H), 7.50-7.22 (m, 3H), 6.90 (brs, 2H), 3.60 (m, 1H), 3.12 (s, 3H), 2.80-2.78 (m, 4H), 1.82-1.70 (m, 4H); MS: 316 (M+H)⁺; m.p.: 212-214° C.

### Example 32

### 1-(1-Acetyl-piperidin-4-yl)-3-(3-fluoro-phenyl)-urea (32)

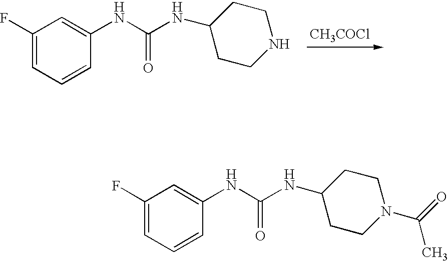

To a solution of 1-(3-fluorophenyl)-3-(piperidin-4-yl)urea (200 mg, 0.841 mmol) in 20 mL of dry dichloromethane was added triethylamine (85 mg, 0.844 mmol). The mixture was stirred at 0° C. for 10 minutes. Acetyl chloride (66 mg, 0.840 mmol) was then added slowly and the reaction mixture was allowed to stir at room temperature for another 2 hours. After completion of the reaction, the mixture was poured into 50 mL of water, extracted with dichloromethane. The organic extract was and washed with saturated sodium bicarbonate solution and brine, and evaporated. The residue was purified by flash column chromatography on silica gel (120-240 mesh) with ethyl acetate/hexane (1:4) as the eluent (R_{f}=0.55) to give 155 mg of 1-(1-acetyl-piperidin-4-yl)-3-(3-fluoro-phenyl)-urea. ¹H NMR (200 MHz, CDCl₃): δ 7.65 (s, 1H), 7.50-7.22 (m, 3H), 6.90 (brs, 2H), 3.60 (m, 1H), 2.80-2.78 (m, 4H), 2.12 (s, 3H), 1.82-1.70 (m, 4H); MS: 280 (M+H)⁺; m.p.: 112-114° C.

### Example 33

### 1-[1-(2-1H-Imidazol-4-yl-acetyl)-piperidin-4-yl]-3-(4-trifluoromethyl-phenyl)-urea (33)

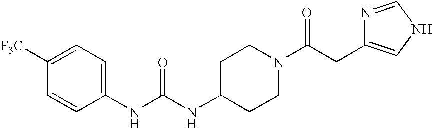

The titled compound was synthesized as a pale yellow solid from 1-(piperidin-4-yl)-3-(4-(trifluoromethyl)phenyl)urea using procedures similar to those described in the above examples. MS: 386 [M+H]⁺; ¹H NMR (300 MHz, DMSO-d₆): δ 1.2-1.3 (m, 2H, CH₂), 1.8-2.0 (d, 2H, CH₂), 3.1-3.5 (m, 4H, CH₂), 2.9-3.0 (tr, H, CH₂), 3.9-4.1 (brs, H, CH), 3.7 (s, 2H, CH₂), 4.4 (d, 2H, CH₂), 6.9 (s, 1H, CH), 7.6 (brs, 4H, Ar—CH), 11.9 (brs, 4H, Ar—CH), 8.8 & 6.2 (brs, d, 2H, NH); m.p.: 121-124° C.; LCMS purity: 91.45%; yield: 29.8%.

### Example 34

### 1-(4-Chloro-phenyl)-3-[1-(2-1H-imidazol-4-yl-acetyl)-piperidin-4-yl]-urea (34)

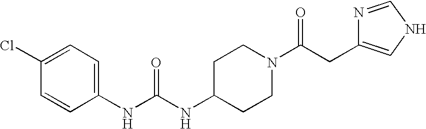

The titled compound was synthesized as a pale yellow solid from readily available starting materials using procedures similar to those described in the above examples. MS: 362 [M+H]⁺; ¹H NMR (300 MHz, DMSO-d₆+CDCl₃): δ1.5-1.6 (m, 2H, CH₂), 1.8-2.2 (d, 2H, CH₂), 3.6-3.8 (m, 2H, CH₂), 3.1-3.4 (m, 2H, CH₂), 3.9-4.1 (brs, 2H, CH₂), 4.4 (m, 1H, CH), 6.9 (brs, 1H, CH), 7.6 (brs, 1H, CH), 7.3-7.5 (d, 4H, Ar—CH), 8.1 & 5.9 (brs, d, 2H, NH); m.p.: 174-176° C.; LCMS purity: 90.56%; yield: 32.8%.

### Example 35

### 1-[1-(1-Methyl-1H-imidazole-4-carbonyl)-piperidin-4-yl]-3-(4-trifluoromethyl-phenyl)-urea (35)

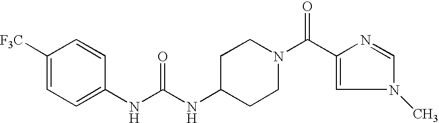

The titled compound was synthesized as a white solid from 1-(piperidin-4-yl)-3-(4-(trifluoromethyl)phenyl)urea using procedures similar to those described in the above examples. MS: 396 [M+H]⁺; ¹H NMR (300 MHz, DMSO-d₆): δ 1.2-1.4 (m, 2H, CH₂), 1.8-2.0 (d, 2H, CH₂), 4.1-4.3 (m, 2H, CH₂), 4.7-4.9 (m, 2H, CH₂), 3.7 (brs, 2H, CH₂), 3.8-3.9 (m, 1H, CH), 7.6-7.8 (m, 4H, Ar—CH), 9.2 & 6.9 (brs, 2H, NH); m.p.: 222-226° C.; LCMS purity: 93.64%; yield: 30.5%.

The following Examples 36-42 were synthesized using procedures similar to those described in the above examples.

### Example 36

### 1-(4-Chlorophenyl)-3-(1-(4-morpholinobenzoyl)piperidin-4-yl)urea (36)

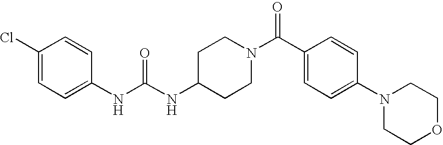

MS: 443 (M+H)⁺; m.p.: 303-308° C.

### Example 37

### 1-(1-(4-Morpholinobenzoyl)piperidin-4-yl)-3-(4-(trifluoromethyl)phenyl)urea (37)

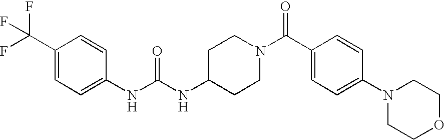

MS: 477 (M+H)⁺; m.p.: 312-315° C.

### Example 38

### Tert-butyl 2-methyl-2-(4-(4-(3-(4-(trifluoromethyl)phenyl)ureido)piperidine-1-carbonyl)phenoxy)propanoate (38)

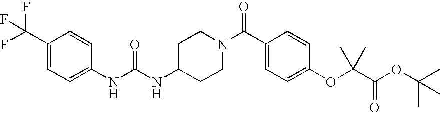

MS: 550 (M+H)⁺; m.p.: 206-208° C.

### Example 39

### 1-(1-(2,5-Dimethyloxazole-4-carbonyl)piperidin-4-yl)-3-(4-(trifluoromethyl)phenyl)urea (39)

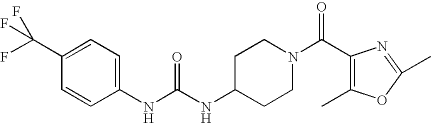

MS: 411 (M+H)⁺; m.p.: 217-219° C.

### Example 40

### 2-Methyl-2-(4-(4-(3-(4-(trifluoromethyl)phenyl)ureido)piperidine-1-carbonyl)phenoxy)propanoic acid (40)

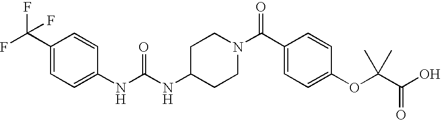

MS: 494 (M+H)⁺; m.p.: 197-199° C.

### Example 41

### 1-(1-Pivaloylpiperidin-4-yl)-3-(4-(trifluoromethyl)phenyl)urea (41)

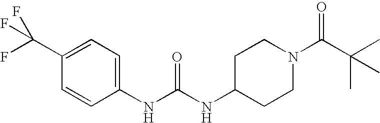

MS: 372 (M+H)⁺; m.p.: 263-265° C.

### Example 42

### 1-(1-(Isopropylsulfonyl)piperidin-4-yl)-3-(4-(trifluoromethyl)phenyl)urea (42)

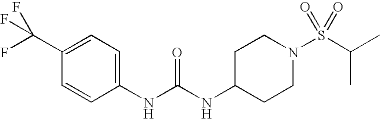

MS: 394 (M+H)⁺; m.p.: 255-258° C.

### Examples 43-54

| | | | LC purity | |
|---|---|---|---|---|
| No. | Structure | (M + H)+ | (%) | M.P.(° C.) |
| 43 | 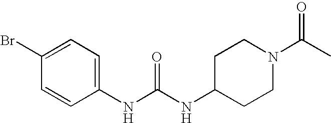 | 341 | 99.3 | 213-216 |
| 44 | 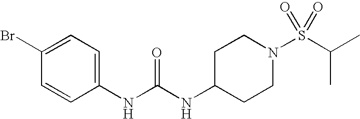 | 405 | 97.9 | 240-242 |
| 45 | 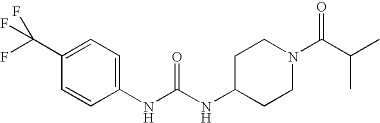 | 358 | 97.8 | 235-236 |
| 46 | 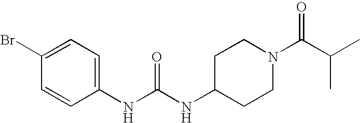 | 369 | 98.18 | 193-204 |
| 47 | 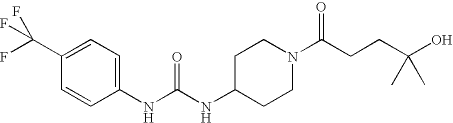 | 402 | 98 | 184-186 |
| 48 | 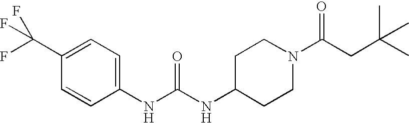 | 386 | 99 | 209-211 |
| 49 | 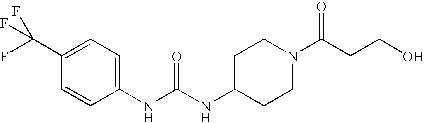 | 360 | 87.8 | 158-160 |
| 50 | 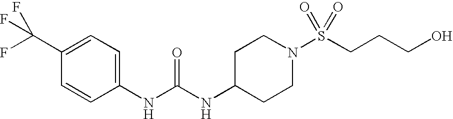 | 410 | 97.9 | 200-205 |
| 51 | 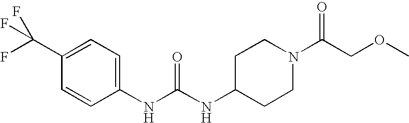 | 360 | 99.3 | 214-215 |
| 52 | 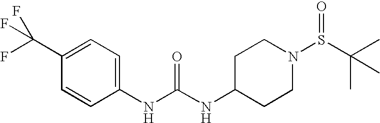 | 392 | 99 | 229-230 |
| 53 | 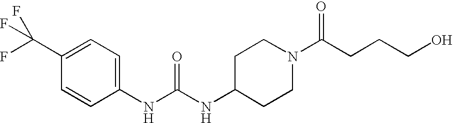 | 374 | 93.3 | 175-182 |
| 54 | 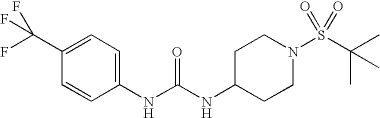 | 408 | 95.5 | 260-261 |
| 55 | 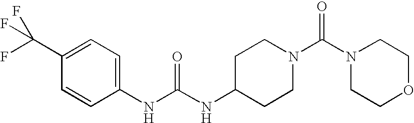 | 401 | 99 | 234-236 |

### BIOLOGICAL EXAMPLES

### Example 1

### Fluorescent Assay for Mouse and Human Soluble Epoxide Hydrolase

Recombinant mouse sEH (MsEH) and human sEH (HsEH) were produced in a baculovirus expression system as previously reported. Grant et al., J. Biol. Chem., 268:17628-17633 (1993); Beetham et al., Arch. Biochem. Biophys., 305:197-201 (1993). The expressed proteins were purified from cell lysate by affinity chromatography. Wixtrom et al., Anal. Biochem., 169:71-80 (1988). Protein concentration was quantified using the Pierce BCA assay using bovine serum albumin as the calibrating standard. The preparations were at least 97% pure as judged by SDS-PAGE and scanning densitometry. They contained no detectable esterase or glutathione transferase activity which can interfere with the assay. The assay was also evaluated with similar results in crude cell lysates or homogenate of tissues.

The IC₅₀s for each inhibitor were determined according to the following procedure:

### Substrate:

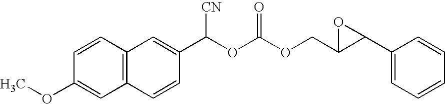

Cyano(2-methoxynaphthalen-6-yl)methyl (3-phenyloxiran-2-yl)methyl carbonate (CMNPC; Jones P. D. et al.; Analytical Biochemistry 2005; 343: pp. 66-75)

### Solutions:

Bis/Tris HCl 25 mM pH 7.0 containing 0.1 mg/mL of BSA (buffer A)

CMNPC at 0.25 mM in DMSO.

Mother solution of enzyme in buffer A (Mouse sEH at 6 μg/mL and Human sEH at 5 μg/mL).

Inhibitor dissolved in DMSO at the appropriate concentration.

### Protocol:

In a black 96 well plate, fill all the wells with 150 μL of buffer A.

Add 2 μL of DMSO in well A2 and A3, and then add 2 μL of inhibitor solution in A1 and A4 through A12.

Add 150 μL of buffer A in row A, then mix several time and transfer 150 μL to row B. Repeat this operation up to row H. The 150 μL removed from row H is discarded.

Add 20 μL of buffer A in column 1 and 2, then add 20 μL of enzyme solution to column 3 to 12.

Incubate the plate for 5 minutes in the plate reader at 30° C.

During incubation prepare the working solution of substrate by mixing 3.68 mL of buffer A (4×0.920 mL) with 266 μL (2×133 μL) of substrate solution).

At t=0, add 30 μL of working substrate solution with multi-channel pipette labeled “Briggs 303” and start the reading ([S]_{final}: 5 μM).

Read with ex: 330 nm (20 nm) and em: 465 nm (20 nm) every 30 second for 10 minutes. The velocities are used to analyze and calculate the IC₅₀s.

Table 2 shows the activity of Compounds 1-55 when tested with the assay at 50 to 5000 nM.

TABLE 2

| | | Conc | % |
|---|---|---|---|
| No. | Structure | (nM) | Inhibition |
| ComparativeExample 1 | 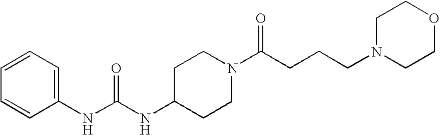 | 5000 | 96 |
| 2 | 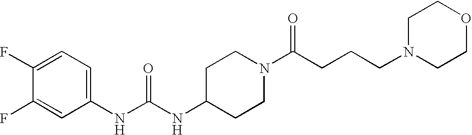 | 500 | 70 |
| 3 | 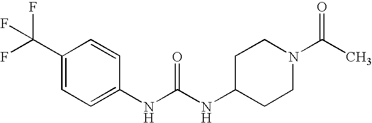 | 50 | 81 |
| 4 | 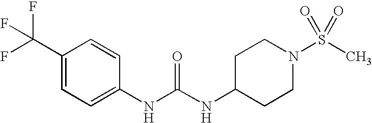 | 50 | 85 |
| ComparativeExample 5 | 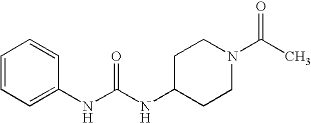 | 5000 | 80 |
| 6 | 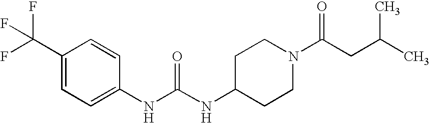 | 50 | 94 |
| 7 | 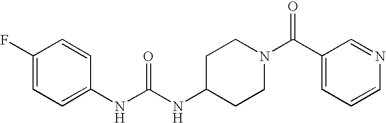 | 500 | 89 |
| 8 | 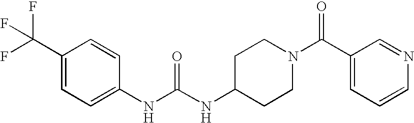 | 50 | 94 |
| 9 | 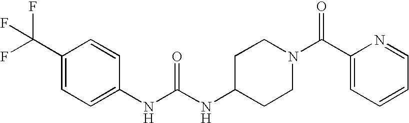 | 50 | 94 |
| 10 | 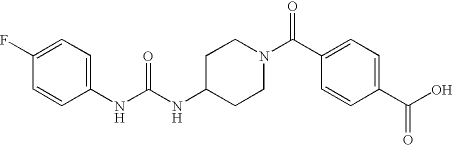 | 50 | 75 |
| 11 | 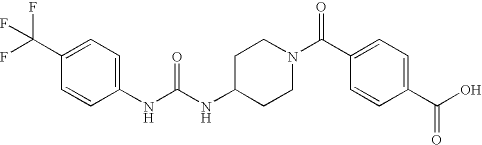 | 50 | 98 |
| 12 | 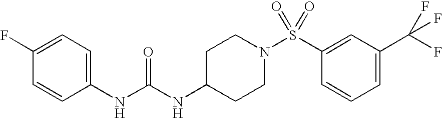 | 50 | 90 |
| 13 | 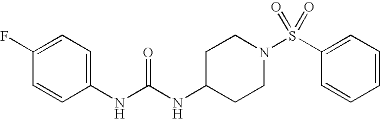 | 50 | 79 |
| 14 | 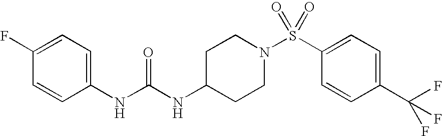 | 50 | 89 |
| 15 | 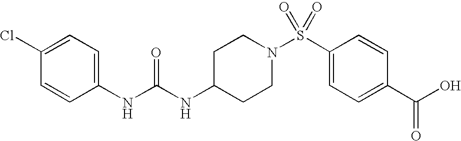 | 50 | 75 |
| 16 | 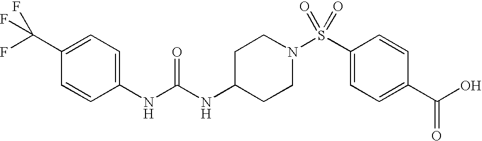 | 50 | 80 |
| 17 | 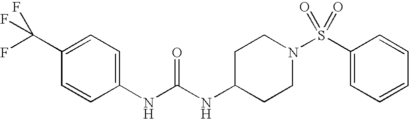 | 50 | 90 |
| 18 | 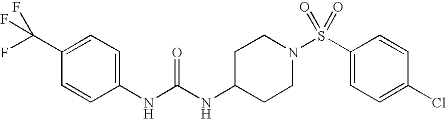 | 50 | 90 |
| 19 | 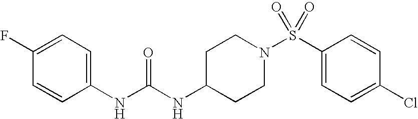 | 500 | 85 |
| 20 | 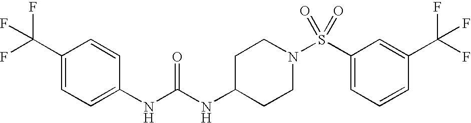 | 50 | 96 |
| 21 | 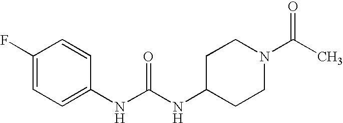 | 500 | 74 |
| 22 | 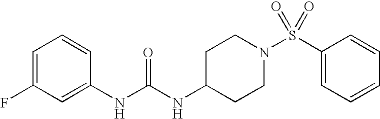 | 50 | 82 |
| 23 | 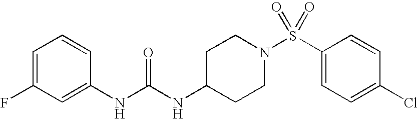 | 50 | 91 |
| 24 | 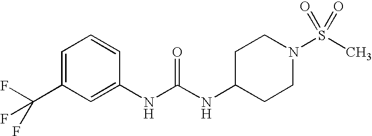 | 50 | 86 |
| 25 | 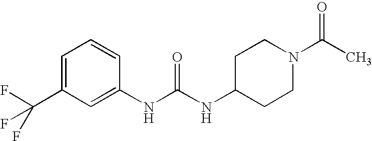 | 50 | 68 |
| 26 | 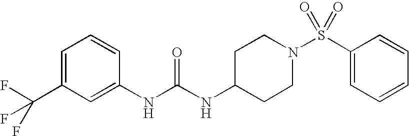 | 50 | 90 |
| 27 | 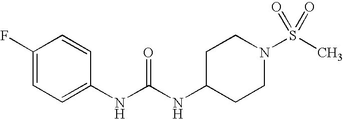 | 50 | 80 |
| 28 | 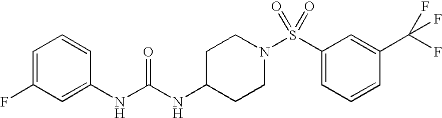 | 50 | 94 |
| 29 | 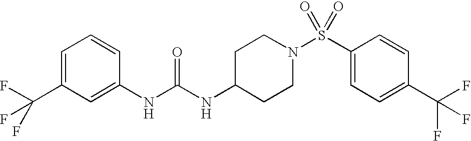 | 50 | 95 |
| 30 | 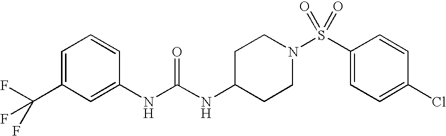 | 50 | 90 |
| 31 | 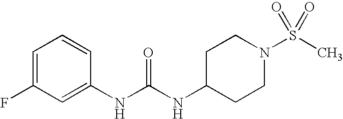 | 500 | 80 |
| 32 | 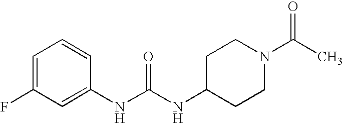 | 500 | 80 |
| 33 | 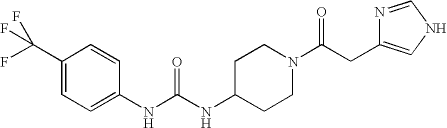 | 50 | 72 |
| 34 | 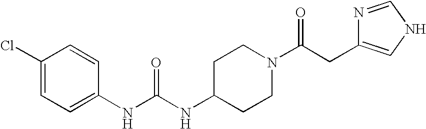 | 500 | 80 |
| 35 | 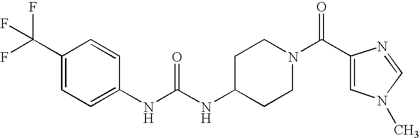 | 50 | 89 |
| 36 | 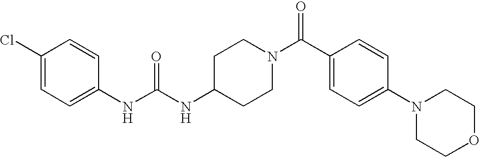 | 50 | 95 |
| 37 | 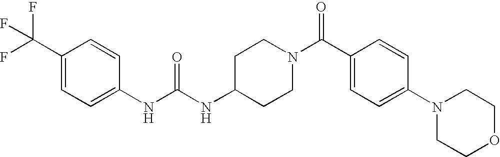 | 50 | 98 |
| 38 | 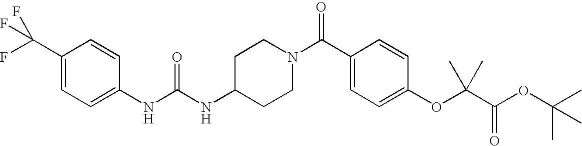 | 50 | 89 |
| 39 | 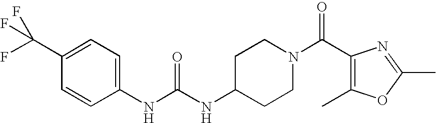 | 50 | 92 |
| 40 | 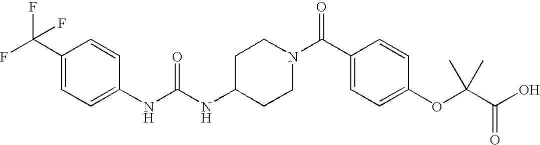 | 50 | 68 |
| 41 | 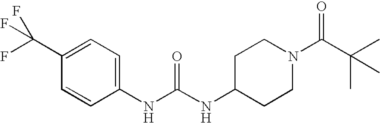 | 50 | 97 |
| 42 | 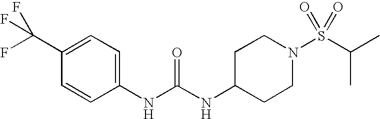 | 50 | 93 |
| 43 | 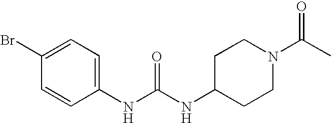 | 200 | 69 |
| 44 | 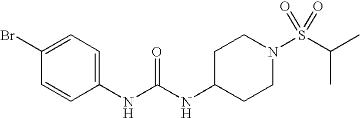 | 200 | 93 |
| 45 | 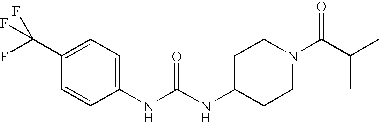 | 200 | 97 |
| 46 | 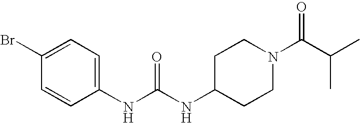 | 200 | 94 |
| 47 | 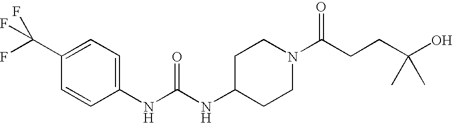 | 200 | 93 |
| 48 | 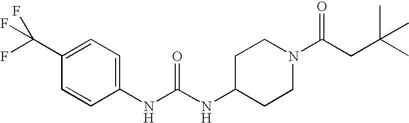 | 200 | 100 |
| 49 | 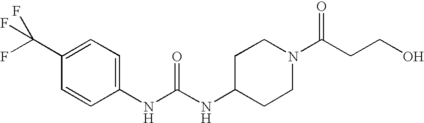 | 200 | 90 |
| 50 | 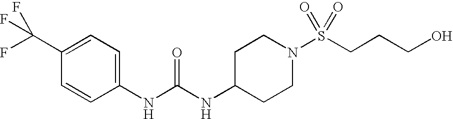 | 200 | 98 |
| 51 | 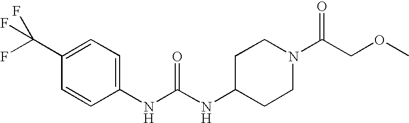 | 2000 | 100 |
| 52 | 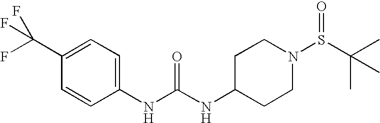 | 200 | 94 |
| 53 | 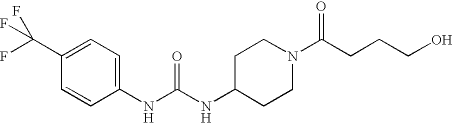 | 200 | 100 |
| 54 | 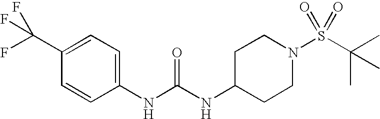 | 200 | 97 |
| 55 | 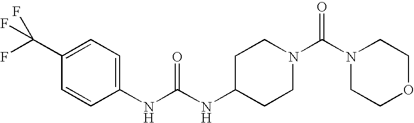 | 2000 | 43 |

The above data demonstrates that inclusion of a halo group onto the phenyl moiety of the phenylureapiperidine imparts significant enhancement of activity. For example, compare the inhibition data for comparative example 1 versus example 2 as well as comparative example 5 versus examples 3 and 4. The above data further demonstrates that the addition of a halo group on the phenylureapiperidine permits significant flexibility in the XY moiety of the compounds of formula I.

### FORMULATION EXAMPLES

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound of the invention | 200 |
| Lactose, spray-dried | 148 |
| Magnesium stearate | 2 |

### Example 3

### Suspension Formulation

The following ingredients are mixed to form a suspension for oral administration (q.s.=sufficient amount).

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |
| distilled water | q.s. to 100 | mL |

### Example 4

### Injectable Formulation

The following ingredients are mixed to form an injectable formulation.

| Ingredient | Quantity per tablet, mg |
|---|---|
| Compound of the invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound of the invention | 500 mg |
| Witepsol ® H-15 | Balance |

What is claimed is:

1. A compound of formula I: 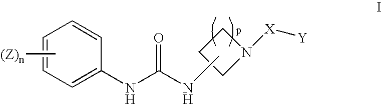
wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3;
or a stereoisomer, tautomer, or pharmaceutically acceptable salt thereof,
provided that when X is C═O and (Z)ₙ is 4-fluoro, Y is not methyl or ethoxy,
provided that when X is SO₂ and (Z)ₙ is 3-fluoro, Y is not 4-tert-butylphenyl, 4-acetylphenyl, 3-methylesterphenyl, or 4-acetylaminophenyl, and
provided that 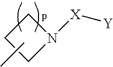
is not 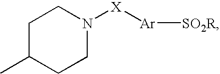
wherein X is as defined herein, Ar is arylene, substituted arylene, heteroarylene or substituted heteroarylene, and R is amino or substituted amino.

2. A compound of formula II: 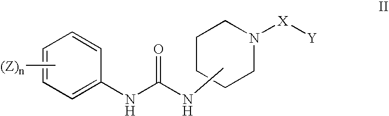
wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl; and
n is an integer equal to 1, 2, or 3;
or a stereoisomer, tautomer, or pharmaceutically acceptable salt thereof,
provided that when X is C═O and (Z)ₙ is 4-fluoro, Y is not methyl or ethoxy,
provided that when X is SO₂ and (Z)ₙ is 3-fluoro, Y is not 4-tert-butylphenyl, 4-acetylphenyl, 3-methylesterphenyl, or 4-acetylaminophenyl, and
provided that 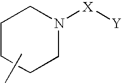
is not 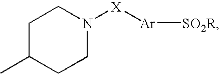
wherein X is as defined herein, Ar is arylene, substituted arylene, heteroarylene or substituted heteroarylene, and R is amino or substituted amino.

3. The compound of claim 2 wherein n is an integer equal to 1 or 2.

4. The compound of claim 2 which is represented by formula III: 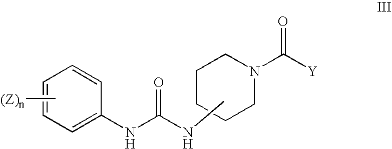
wherein:
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl; and
n is an integer equal to 1, 2, or 3;
or a stereoisomer, tautomer, or pharmaceutically acceptable salt thereof,
provided that 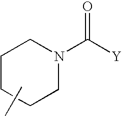
is not 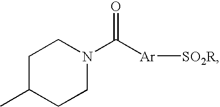
wherein Ar is arylene, substituted arylene, heteroarylene or substituted heteroarylene, and R is amino or substituted amino.

5. The compound of claim 4 wherein n is an integer equal to 1 to 2.

6. The compound of claim 2 which is represented by formula IV: 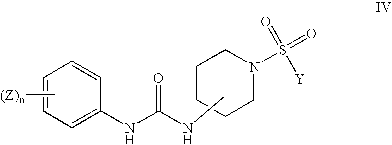
wherein:
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl; and
n is an integer equal to 1, 2, or 3;
or a stereoisomer, tautomer, or pharmaceutically acceptable salt thereof,
provided that 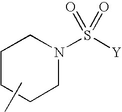
is not 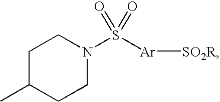
wherein Ar is arylene, substituted arylene, heteroarylene or substituted heteroarylene, R is amino or substituted amino.

7. The compound of claim 6 wherein n is an integer equal to 1 to 2.

8. The compound of claim 2 which is represented by 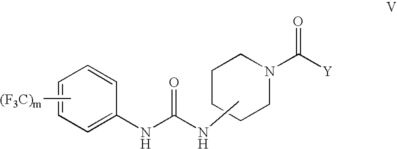
wherein:
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
m is 1 or 2;
or a stereoisomer, tautomer, or pharmaceutically acceptable salt thereof
provided that 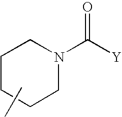
is not 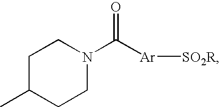
wherein Ar is arylene, substituted arylene, heteroarylene or substituted heteroarylene, and R is amino or substituted amino.

9. The compound of claim 2 which is represented by 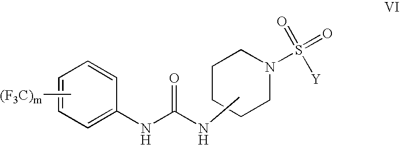
wherein:
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
m is 1 or 2;
or a stereoisomer, tautomer, or pharmaceutically acceptable salt thereof,
provided that 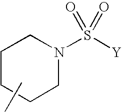
is not 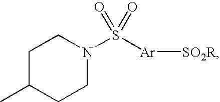
wherein Ar is arylene, substituted arylene, heteroarylene or substituted heteroarylene, R is amino or substituted amino.

10. The compound of claim 2 wherein Y is phenyl or substituted phenyl.

11. The compound of claim 2 wherein Y is pyridinyl or substituted pyridinyl.

12. The compound of claim 2 wherein Y is morpholinyl or morpholinyl-(C₁-C₃)alkyl.

13. The compound of claim 2 wherein Y is imidazolyl, (C₁-C₃)methylimidazolyl, or imidazolyl-(C₁-C₃)alkyl.

14. The compound of claim 2 wherein group 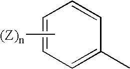
represents 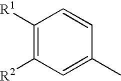
wherein R¹ and R² are independently selected from the group consisting of hydrogen, halogen, and trifluoromethyl.

15. The compound of claim 14 wherein R¹ and R² are trifluoromethyl.

16. The compound of claim 14 wherein R¹ and R² are fluorine.

17. The compound of claim 14 wherein R¹ and R² are independently trifluoromethyl and hydrogen.

18. The compound of claim 14 wherein R¹ and R² are independently halogen and hydrogen.

19. The compound of claim 14 wherein X is C═O or SO₂, Y is phenyl or substituted phenyl, and R¹ and R² are independently selected from the group consisting of fluoro and trifluoromethyl.

20. The compound of claim 14 wherein X is C═O or SO₂, Y is pyridinyl or substituted pyridinyl, and R¹ and R² are independently selected from the group consisting of fluoro and trifluoromethyl.

21. The compound of claim 14 wherein X is C═O or SO₂, Y is imidazolyl, (C₁-C₃)alkyl-imidazolyl, or imidazoly-(C₁-C₃)alkyl, and R¹ and R² are independently selected from the group consisting of fluoro and trifluoromethyl.

22. The compound of claim 14 wherein X is C═O or SO₂, Y is morpholinyl, (C₁-C₃)alkyl-morpholinyl, or morpholiny-(C₁-C₃)alkyl, and R¹ and R² are independently selected from the group consisting of fluoro and trifluoromethyl.

23. A compound selected from the group consisting of
1-(3,4-Difluoro-phenyl)-3-[1-(4-morpholin-4-yl-butyryl)-piperidin-4-yl]-urea,
1-(1-Acetyl-piperidin-4-yl)-3-(4-trifluoromethyl-phenyl)-urea,
1-(1-Methanesulfonyl-piperidin-4-yl)-3-(4-trifluoromethyl-phenyl)-urea,
1-[1-(3-Methyl-butyryl)-piperidin-4-yl]-3-(4-trifluoromethyl-phenyl)-urea,
1-(4-Fluoro-phenyl)-3-[1-(pyridine-3-carbonyl)-piperidin-4-yl]-urea,
1-[1-(Pyridine-3-carbonyl)-piperidin-4-yl]-3-(4-trifluoromethyl-phenyl)-urea,
1-[1-(Pyridine-2-carbonyl)-piperidin-4-yl]-3-(4-trifluoromethyl-phenyl)-urea,
4-{4-[3-(4-Fluoro-phenyl)-ureido]-piperidine-1-carbonyl}-benzoic acid,
4-{4-[3-(4-Trifluoromethyl-phenyl)-ureido]-piperidine-1-carbonyl}-benzoic acid,
1-(4-Fluoro-phenyl)-3-[1-(3-trifluoromethyl-benzenesulfonyl)-piperidin-4-yl]-urea,
1-(1-Benzenesulfonyl-piperidin-4-yl)-3-(4-fluoro-phenyl)-urea,
1-(4-Fluoro-phenyl)-3-[1-(4-trifluoromethyl-benzenesulfonyl)-piperidin-4-yl]-urea,
4-{4-[3-(4-Chloro-phenyl)-ureido]-piperidine-1-sulfonyl}-benzoic acid,
4-{4-[3-(4-Trifluoromethyl-phenyl)-ureido]-piperidine-1-sulfonyl}-benzoic acid,
1-(1-Benzenesulfonyl-piperidin-4-yl)-3-(4-trifluoromethyl-phenyl)-urea,
1-[1-(4-Chloro-benzenesulfonyl)-piperidin-4-yl]-3-(4-trifluoromethyl-phenyl)-urea,
1-[1-(4-Chloro-benzenesulfonyl)-piperidin-4-yl]-3-(4-fluoro-phenyl)-urea,
1-[1-(3-Trifluoromethyl-benzenesulfonyl)-piperidin-4-yl]-3-(4-trifluoromethyl-phenyl)-urea,
1-(1-Acetyl-piperidin-4-yl)-3-(4-fluoro-phenyl)-urea,
1-(1-Benzenesulfonyl-piperidin-4-yl)-3-(3-fluoro-phenyl)-urea,
1-[1-(4-Chloro-benzenesulfonyl)-piperidin-4-yl]-3-(3-fluoro-phenyl)-urea,
1-(1-Methanesulfonyl-piperidin-4-yl)-3-(3-trifluoromethyl-phenyl)-urea,
1-(1-Acetyl-piperidin-4-yl)-3-(3-trifluoromethyl-phenyl)-urea,
1-(1-Benzenesulfonyl-piperidin-4-yl)-3-(3-trifluoromethyl-phenyl)-urea,
1-(4-Fluoro-phenyl)-3-(1-methanesulfonyl-piperidin-4-yl)-urea,
1-(3-Fluoro-phenyl)-3-[1-(3-trifluoromethyl-benzenesulfonyl)-piperidin-4-yl]-urea,
1-[1-(4-Trifluoromethyl-benzenesulfonyl)-piperidin-4-yl]-3-(3-trifluoromethyl-phenyl)-urea,
1-[1-(4-Chloro-benzenesulfonyl)-piperidin-4-yl]-3-(3-trifluoromethyl-phenyl)-urea,
1-(3-Fluoro-phenyl)-3-(1-methanesulfonyl-piperidin-4-yl)-urea,
1-(1-Acetyl-piperidin-4-yl)-3-(3-fluoro-phenyl)-urea,
1-[1-(2-1H-Imidazol-4-yl-acetyl)-piperidin-4-yl]-3-(4-trifluoromethyl-phenyl)-urea,
1-(4-Chloro-phenyl)-3-[1-(2-1H-imidazol-4-yl-acetyl)-piperidin-4-yl]-urea,
1-[1-(1-Methyl-1H-imidazole-4-carbonyl)-piperidin-4-yl]-3-(4-trifluoromethyl-phenyl)-urea,
1-(4-Chlorophenyl)-3-(1-(4-morpholinobenzoyl)piperidin-4-yl)urea,
1-(1-(4-Morpholinobenzoyl)piperidin-4-yl)-3-(4-(trifluoromethyl)phenyl)urea,
Tert-butyl 2-methyl-2-(4-(4-(3-(4-(trifluoromethyl)phenyl)ureido)piperidine-1-carbonyl)phenoxy)propanoate,
1-(1-(2,5-Dimethyloxazole-4-carbonyl)piperidin-4-yl)-3-(4-(trifluoromethyl)phenyl)urea,
2-Methyl-2-(4-(4-(3-(4-(trifluoromethyl)phenyl)ureido)piperidine-1-carbonyl)phenoxy)propanoic acid,
1-(1-Pivaloylpiperidin-4-yl)-3-(4-(trifluoromethyl)phenyl)urea, and
1-(1-(Isopropylsulfonyl)piperidin-4-yl)-3-(4-(trifluoromethyl)phenyl)urea,
1-(1-acetylpiperidin-4-yl)-3-(4-bromophenyl)urea,
1-(4-bromophenyl)-3-(1-(isopropylsulfonyl)piperidin-4-yl)urea,
1-(1-isobutyrylpiperidin-4-yl)-3-(4-(trifluoromethyl)phenyl)urea,
1-(4-bromophenyl)-3-(1-isobutyrylpiperidin-4-yl)urea,
1-(1-(4-hydroxy-4-methylpentanoyl)piperidin-4-yl)-3-(4-(trifluoromethyl)phenyl)urea,
1-(1-(3,3-dimethylbutanoyl)piperidin-4-yl)-3-(4-(trifluoromethyl)phenyl)urea,
1-(1-(3-hydroxypropanoyl)piperidin-4-yl)-3-(4-(trifluoromethyl)phenyl)urea,
1-(1-(3-hydroxypropylsulfonyl)piperidin-4-yl)-3-(4-(trifluoromethyl)phenyl)urea,
1-(1-(2-methoxyacetyl)piperidin-4-yl)-3-(4-(trifluoromethyl)phenyl)urea,
1-(1-(4-hydroxybutanoyl)piperidin-4-yl)-3-(4-(trifluoromethyl)phenyl)urea, and
1-(1-(tert-butylsulfonyl)piperidin-4-yl)-3-(4-(trifluoromethyl)phenyl)urea,
or a stereoisomer, tautomer, or pharmaceutically acceptable salt thereof.

24. A compound of formula VII or a stereoisomer, tautomer, or pharmaceutically acceptable salt thereof: 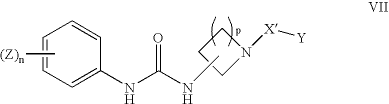
wherein:
X′ is S or SO;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3.

25. A compound of claim 24 of formula VIII or a stereoisomer, tautomer, or pharmaceutically acceptable salt thereof: 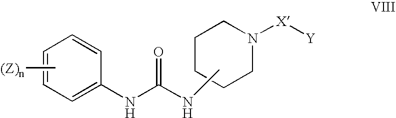
wherein:
X′ is S or SO;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3.

26. A compound of claim 24 which is 1-(1-(tert-butylsulfinyl)piperidin-4-yl)-3-(4-(trifluoromethyl)phenyl)urea.

27. A pharmaceutical composition comprising a pharmaceutically acceptable carrier and a therapeutically effective amount of a compound of claim 1 or claim 24 for treating a soluble epoxide hydrolase mediated disease.

28. A method for treating a soluble epoxide hydrolase mediated disease, comprising the step of administering to a patient a pharmaceutical composition comprising a pharmaceutically acceptable carrier and a therapeutically effective amount of a compound of formula I or a stereoisomer, or pharmaceutically acceptable salt thereof: 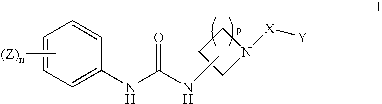
wherein:
X is C═O or SO₂;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl; and
n is an integer equal to 1, 2, or 3,
p is an integer equal to 1, 2, or 3,
provided that when X is C═O and (Z)ₙ is 4-fluoro, Y is not methyl or ethoxy, and
provided that when X is SO₂ and (Z)ₙ is 3-fluoro, Y is not 4-tert-butylphenyl, 4-acetylphenyl, 3-methylesterphenyl, or 4-acetylaminophenyl, and
provided that 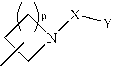
is not 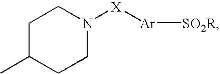
wherein X is as defined herein, Ar is arylene, substituted arylene, heteroarylene or substituted heteroarylene, and R is amino or substituted.

29. A method for treating a soluble epoxide hydrolase mediated disease, comprising the step of administering to a patient a pharmaceutical composition comprising a pharmaceutically acceptable carrier and a therapeutically effective amount of a compound of formula VII or a stereoisomer, or pharmaceutically acceptable salt thereof: 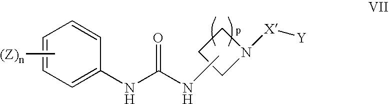
wherein:
X′ is S or SO;
Y is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
Z is independently selected from the group consisting of halogen and haloalkyl;
n is an integer equal to 1, 2, or 3; and
p is an integer equal to 1, 2, or 3.
